SEQUENCE LISTING (19 sequences)

SEQ ID NO: 1 - DNA, 1044 bp, Homo sapien
gcggctcggggacccgtagagcccggcgctgcgcgcatggccctgctctcgcgccccgcgctcaccctcctgctcctcctcatggccgctgttgtcaggtgccaggagcaggcccagaccaccgactggagagccaccctgaagaccatccggaacggcgttcataagatagacacgtacctgaacgccgccttggacctcctgggaggcgaggacggtctctgccagtataaatgcagtgacggatctaagcctttcccacgttatggttataaaccctccccaccgaatggatgtggctctccactgtttggtgttcatcttaacattggtatcccttccctgacaaagtgttgcaaccaacacgacaggtgctatgaaacctgtggcaaaagcaagaatgactgtgatgaagaattccagtattgcctctccaagatctgccgagatgtacagaaaacactaggactaactcagcatgttcaggcatgtgaaacaacagtggagctcttgtttgacagtgttatacatttaggttgtaaaccatatctggacagccaacgagccgcatgcaggtgtcattatgaagaaaaaactgatctttaaaggagatgccgacagctagtgacagatgaagatggaagaacataacctttgacaaataactaatgtttttacaacataaaactgtcttatttttgtgaaaggattattttgagaccttaaaataatttatatcttgatgttaaaacctcaaagcaaaaaaagtgagggagatagtgaggggagggcacgcttgtcttctcaggtatcttccccagcattgctcccttacttagtatgccaaatgtcttgaccaatatcaaaaacaagtgcttgtttagcggagaattttgaaaagaggaatatataactcaattttcacaaccacatttaccaaaaaaagagatcaaatataaaattcatcataatgtctgttcaacattatcttatttggaaaatggggaaattatcacttacaagtatttgtttactatgagattt

SEQ ID NO: 2 - protein, 189 aa, Homo sapien
MALLSRPALTLLLLLMAAVVRCQEQAQTTDWRATLKTIRNGVHKIDTYLNAALDLLGGEDGLCQYKCSDGSKPFPRYGYKPSPPNGCGSPLFGVHLNIGIPSLTKCCNQHDRCYETCGKSKNDCDEEFQYCLSKICRDVQKTLGLTQHVQACETTVELLFDSVIHLGCKPYLDSQRAACRCHYEEKTDL

SEQ ID NO: 3 - DNA, 1529 bp, Mus musculus
gagctcgcgagcgcggaggaggccggggtcctgagccggagccggagcgcgcgccgctgcccagccccgccgcgccggccccgcagatggtgactccgcggcccgcgcccgcccggggccccgcgctcctcctcctcctgctgctggccactgcgcgcgggcaggaacaggaccagaccaccgactggagggccaccctcaagaccatccgcaacggcatccacaagatagacacgtacctcaacgccgcgctggacctgctgggcggggaggacgggctctgccagtacaagtgcagcgacggatcgaagcctgttccacgctatggatataaaccatctccaccaaatggctgtggctctccactgtttggcgttcatctgaacataggtatcccttccctgaccaagtgctgcaaccagcacgacagatgctatgagacctgcgggaaaagcaagaacgactgtgacgaggagttccagtactgcctctccaagatctgcagagacgtgcagaagacgctcggactatctcagaacgtccaggcatgtgagacaacggtggagctcctctttgacagcgtcatccatttaggctgcaagccatacctggacagccagcgggctgcatgctggtgtcgttatgaagaaaaaacagatctataaagaccctgactgctggagagcaggcgagaatggaggatcatccttgccaaagatcggatgctttaacagcctaatgttgccttagttttgtgtcgatgggtcattttgagacctttctatactgtgtctttttttagaacctcaaagtgaaaacggtggggggccaggcagaaacagagggagagcatgcttgggatggggagcgagcagacatccaagagcatgccttcctgagactcgctgtcttggtggctcccccaaactgggaagaaaagcttaagctcgtgtgacttggtgttcatagttgtacttaacaataaaaatgaaagcaaatgtaaaattcattgtaaggacttttcagcattattttattttgaaatacaggccaatcttcccttagaactattatttattttgaaatttcagatgtacatttatacctggaaaaactattaattctccatttttattatacataatgtgttgtttctctgaagcccactaagataggtataaatatgttactcaaaactacacggtttccaaatgtgcatctcttgtacagttggaatcacggttggtacttctctggagagacgccccaggacatctgagtgttgggatgtgcacagaattcagaagcccagcttcctgtctcacaaaccgcttagagtgaatatccttcctctcctgctgtgagctctaggaatgacgggtttaacgggccaagccgagctctgaatcagtgcgctatctgctgctgaggttgtggttactccctcatccccgttttccatcttctatcctggagtagtgttaaaagtctgacattttctaatggaggtcttaataaaagctatttac

SEQ ID NO: 4 - protein, 192 aa, Mus musculus
MVTPRPAPARGPALLLLLLLATARGQEQDQTTDWRATLKTIRNGIHKIDTYLNAALDLLGGEDGLCQYKCSDGSKPVPRYGYKPSPPNGCGSPLFGVHLNIGIPSLTKCCNQHDRCYETCGKSKNDCDEEFQYCLSKICRDVQKTLGLSQNVQACETTVELLFDSVIHLGCKPYLDSQRAACWCRYEEKTDL

SEQ ID NO: 5 - DNA, 476 bp, Mus musculus
ctagacacaaaaaatgaaagactaccacagtggtcctgggaagtactgggagccatttgcctttcctgtaggctgttccgggacagaagaagaagaaggtctcaggattggaagatcgaagcctgttccacgctatggatataaaccatctccaccaaatggctgtggctcgccactgtttggcgttcatctgaacataggtatcccttccctgaccaagtgctgcaaccagcacgacagatgctacgagacctgcgggaaaagcaagaacgactgtgacgaggagttccagtactgcctctccaagatctgcagagacgtgcagaagacgctcggactatctcagaacgtccaggcatgtgagacaacggtggagctcctctttgacagcgtcatccatttaggctgcaagccatacctggacagccagcgggctgcatgctggtgtcgttatgaagaaaaaacagatctataaa

SEQ ID NO: 6 - protein, 153 aa, Mus musculus
MKDYHSGPGKYWEPFAFPVGCSGTEEEEGLRIGRSKPVPRYGYKPSPPNGCGSPLFGVHLNIGIPSLTKCCNQHDRCYETCGKSKNDCDEEFQYCLSKICRDVQKTLGLSQNVQACETTVELLFDSVIHLGCKPYLDSQRAACWCRYEEKTDL

SEQ ID NO: 7 - protein, 146 aa, Mus musculus
MKLLLLAALLTAGAAAHSISPRAVWQFRNMIKCTIPGSDPLKDYNNYGCYCGLGGWGTPVDDLDRCCQTHDHCYSQAKKLESCKFLIDNPYTNTYSYSCSGSEITCSAKNNKCEDFICNCDREAAICFSKVPYNKEYKNLDTGKFC

SEQ ID NO: 8 - protein, 146 aa, Mus musculus
MKVLLLLAASIMAFGSIQVQGNIAQFGEMIRLKTGKRAELSYAFYGCHCGLGGKGSPKDATDRCCVTHDCCYKSLEKSGCGTKLLKYKYSHQGGQITCSANQNSCQKRLCQCDKAAAECFARNKKTYSLKYQFYPNMFCKGKKPKC

SEQ ID NO: 9 - protein, 137 aa, Mus musculus
MKGLLTLAWFLACSVPAVPGGLLELKSMIEKVTRKNAFKNYGFYGCYCGWGGRGTPKDGTDWCCQMHDRCYGQLEEKDCAIRTQSYDYRYTNGLVICEHDSFCPMRLCACDRKLVYCLRRNLWTYNPLYQYYPNFLC

SEQ ID NO: 10 - protein, 143 aa, Mus musculus
MLLLLLLLLLGPGPGFSEATRRSHVYKRGLLELAGTLDCVGPRSPMAYMNYGCYCGLGGHGEPRDAIDWCCYHHDCCYSRAQDAGCSPKLDRPRSPMAYMNYGCYCGLGGHGEPRDAIDWCCYHHDCCYSRAQDAGCSPKLDR

SEQ ID NO: 11 - DNA, 25 bp, Mus musculus
gggcaggaacaggaccagaccaccg

SEQ ID NO: 12 - DNA, 26 bp, Mus musculus
ccacagtggtcctgggaagtactggg

SEQ ID NO: 13 - DNA, 31 bp, Mus musculus
ggtttatatccatagcgtggaacaggcttcg

SEQ ID NO: 14 - DNA, 22 bp, Mus musculus
gtccatgattgagaaggtgacc

SEQ ID NO: 15 - DNA, 22 bp, Mus musculus
tcataggactgggtccgaatgg

SEQ ID NO: 16 - DNA, 22 bp, Mus musculus
ggtcacatgtatacaagcgtgg

SEQ ID NO: 17 - DNA, 22 bp, Mus musculus
atgtgatggtccatgcacttcc

SEQ ID NO: 18 - DNA, 20 bp, Mus musculus
gtgggccgctctaggcacca

SEQ ID NO: 19 - DNA, 25 bp, Mus musculus
cggttggccttagggttcagggggg

(19) United States
(12) Patent Application Publication
(10) Pub. No.: US 2002/0110891 A1
(43) Pub. Date: Aug. 15, 2002

(54) Phospholipase A2 group preferentially expressed in TH2 cells

(75) Inventors: I-Cheng Ho, Newton, MA (US); Jonathan P. Arm, Brookline, MA (US); K. Frank Austen, Wellesley Hills, MA (US); Laurie H. Glimcher, West Newton, MA (US)

Correspondence Address: LAHIVE & COCKFIELD, 28 STATE STREET, BOSTON, MA, 02109, US

(73) Assignee: President and Fellows of Harvard College, Cambridge, MA

(21) Appl. No.: 09/993,999

(22) Filed: Nov. 6, 2001

Related U.S. Application Data
(60) Provisional application No. 60/246,316, filed on Nov. 6, 2000.

(51) Int. Cl. C12N 9/18; C12Q 1/68; C07H 21/04; C12P 21/02; C12N 5/06

(52) U.S. Cl. 435/197; 435/6; 435/69.1; 435/320.1; 435/325; 536/23.2

(57) ABSTRACT

Isolated nucleic acid molecules encoding a novel phospholipase A₂, GXII PLA₂, that is preferentially expressed in Th2 cells, are disclosed. The invention further provides recombinant expression vectors containing a nucleic acid molecule of the invention, host cells into which the expression vectors have been introduced, antisense nucleic acid molecules, non-human transgenic animals carrying a GXII PLA₂ transgene and non-human transgenic animals deficient in GXII PLA₂. The invention further provides isolated GXII PLA₂ proteins and peptides, GXII PLA₂ fusion proteins and anti-GXII PLA₂ antibodies. Methods of using the GXII PLA₂ compositions of the invention are also disclosed, including methods for detecting GXII PLA₂ protein or mRNA in a biological sample, methods of modulating GXII PLA₂ activity in a cell, and methods for identifying agents that modulate GXII PLA₂ activity. Methods of modulating Th2 cell differentiation or activity by modulating either GXII PLA₂ or GV PLA₂, which is also preferentially expressed in T cells, are provided. Methods for modulating prostaglandin production in Th2 cells by modulating GXII PLA₂ activity or GV PLA₂ activity are also provided.

### RELATED APPLICATIONS

This application claims priority to U.S. Provisional Application No. 60/246,316 filed on Nov. 6, 2000, the contents of which are incorporated herein in their entirety by this reference.

### GOVERNMENT FUNDING

Work described herein was supported, at least in part, under grants Al/AG 37833 and P01 HL361 10 awarded by the National Institutes of Health. The U.S. government therefore may have certain rights in this invention.

### BACKGROUND OF THE INVENTION

The family of phospholipase A₂ (PLA₂) enzymes is defined by the enzymatic activities of its members, which hydrolyze the sn-2 ester bond of phospholipids to release free fatty acids (Dennis, E. A. (1997) Trends in Biochemical Sciences 22:1-2). Based on their protein structure and biochemical properties, mammalian PLA₂ enzymes can be divided into several classes: low molecular weight secretory, 85 kDa cytosolic (cPLA₂), selective acetyl hydrolases of platelet activating factor, and calcium-independent PLA₂ (iPLA₂). Thus far, eight low molecular weight mammalian PLA₂ enzymes have been described including group (G) IB, five GII enzymes, GV and GX (Murakami, M. et al. (1992) J. Biochem. 111:175-181; Valentin, E. et al. (1999) J. Biol. Chem. 274:19152-19160; Cupillard, L. et al. (1997) J. Biol. Chem. 272:15745-15752; Chen, J. et al. (1994) J. Biol. Chem. 269:2365-2368). Each low molecular weight PLA₂ is 13-15 kDa in size and contains a signal peptide and 12-14 cysteine residues, the positions and spacing of which are conserved among the low molecular weight PLA₂ members (Tischfield, J. A. (1997) J. Biol. Chem. 272:17247-17250). Low molecular weight PLA₂ enzymes require millimolar Ca²⁺ for activity and display no specificity for the fatty acid in the sn-2 position of phospholipids (Murakami, M. et al. (1995) Journal of Lipid Mediators & Cell Signalling 12:119-130). cPLA₂, or GIV PLA₂, is a large cytosolic enzyme (˜85 kDa), requires only micromolar Ca²⁺ for activity, and is the only cloned PLA₂ that displays high selectivity toward arachidonate-containing phospholipids (Leslie, C. C. (1997) J. Biol. Chem. 272:16709-16712). The hematopoietic iPLA₂, or GVI PLA₂ is an 85 kDa cytosolic enzyme (Balsinde, J. and Dennis, E. A. (1997) J. Biol. Chem. 272:16069-16072). In addition to the absence of a calcium requirement, the hematopoietic iPLA₂ contains eight ankyrin motifs which are not found in any other PLA₂ (Tang, J. et al. (1997) J. Biol. Chem. 272:8567-8575). Groups IV, VI, and the platelet activating factor acetyl hydrolases (GVII and GVIII) exhibit a catalytic serine, while the cysteine-rich, low molecular weight groups utilize a catalytic histidine.

GIV PLA₂ has been established as essential for providing arachidonic acid to the downstream enzymes, such as cycloxygenase (COX) and lipoxygenase, for generation of prostanoids and leukotrienes by comparison of various hematopoietic cell types derived from mice with disruption of this gene with their normal littermates (Bonventre, J. V. et al. (1997) Nature 390:622-625; Uozumi, N. et al. (1997) Nature 390:618-622; Fujishima, H. et al. (1999) Proc. Natl. Acad. Sci. USA 96:4803-4807). GV PLA₂, though non-selective as to the sn-2 fatty acid, has been implicated as a supporting enzyme for prostanoid biosynthesis in cell systems by the kinetics of transcript induction and effects of generic inhibitors for several related groups (Balsinde, J. et al. (1999) J. Biol. Chem. 274:25967-25970; Shinohara, H. et al. (1999) J. Biol. Chem. 274:12263-12268), but definitive evidence awaits cells with gene disruption. GIIA PLA₂, a secretory granule-stored species has been shown to augment arachidonic acid release by an extracellular membrane action which can be active site dependent or mediated by cell activation through lectin-like receptors (Copic, A. et al. (1999) J. Biol. Chem. 274:26315-26320; Cupillard, L. et al. (1999) J. Biol. Chem. 274:7043-7051). In addition to these downstream eicosanoid mediators, there are metabolites with gene inducing functions via interaction with the intracellular receptors of the PPAR family (Forman, B. M. et al. (1995) Cell 83:803-812; Kliewer, S. A. et al. (1995) Cell 83:813-819). Finally, both released arachidonic acid and the concomitantly generated lysophospholipid have second messenger functions (Berk, P. D. and Stump, D. D. (1999) Mol. Cell. Biochem. 192:17-31; Moolenaar, W. H. et al. (1997) Curr. Opin. Cell Biol. 9:168-173; Balsinde, J. et al. (1997) J. Biol. Chem. 272:29317-29321). None of the available information reveals a particular T cell distribution for any PLA₂ group nor do the limited examples of gene disruption, GIV and GIIA, provide evidence of an anomaly in the adaptive immune response (Bonventre, J. V. et al. (1997) Nature 390:622-625; Uozumi, N. et al. (1997) Nature 390:618-622; Kennedy, B. P. et al. (1995) J. Biol. Chem. 270:22378-22385; MacPhee, M. et al. (1995) Cell 81:957-966).

Several lines of evidence suggest that the PLA₂/COX/prostaglandin cascade might play a critical role in regulating the differentiation and function of helper T cells. CD4+helper T cells can be divided into two functional subsets based on their secreted cytokines (Mosmann, T. R. and Sad, S. (1996) Immunol. Today 17:138-146; Abbas, A. K. et al. (1996) Nature 383:787-793). Type 1 helper (Th1) T cells secrete IFN-γ . and IL-2 and are responsible for delayed type hypersensitivity and eradication of intracellular microorganisms. Type 2 helper (Th2) T cells produce cytokines such as IL-4 and IL-13, which enhance the production of IgE antibodies involved in allergic responses, and IL-5, which promotes the maturation of eosinophils. Furthermore, IL-4 regulates FcεRI expression of human mast cells, while IL-5 is comitogenic with stem cell factor for their mucosal expansion (Bischoff, S. C. et al. (1999) Proc. Natl. Acad. Sci. USA 96:8080-8085). There is evidence that prostaglandin E2 (PGE2), a metabolite of the cycloxygenase pathway, selectively promotes the differentiation of Th2 cells (Hilkens, C. M. et al. (1996) European Respiratory Jounal—Supplement 1996 22:90s-94s). Production of IL-2 and IFN-γ by short-term peripheral blood lymphocyte cultures and by long-term Th clones is inhibited by PGE2 in a dose-dependent fashion, whereas IL-4 and IL-5 production is enhanced (Snijdewint, F. G. et al (1993) J. Immunol. 150:5321-5329). Naive human CD4+T cells stimulated with anti-CD3 in the presence of PGE2 display a Th2 phenotype which is maintained upon restimulation in the absence of PGE2 (Katamura, K. et al. (1995) J. Immunol. 155:4604-4612). Furthermore, PGE2 can directly inhibit transcription of the IL-2 gene, but not the IL-4 gene, in human Jurkat T cells (Paliogianni, F. et al. (1993) J. Exp. Med. 178:1813-1817; Paliogianni, F. and Boumpas, D. T. (1996) Cell. Immunol. 171:95-101). In addition to its direct effect on the transcription of cytokine genes in T cells, PGE2 inhibits the release of IL-12, the potent Th1 promoting cytokine, from antigen presenting cells, such as dendritic cells (Kuroda, E. et al. (2000) J. Immunol. 164:2386-2395; Kalinski, P. et al. (1998) J. Immunol. 161:2804-2809); and inhibits the expression of IL-12 receptor in differentiating Th cells (Wu, C. Y. et al. (1998) J. Immunol. 161:2723-2730). Recently, PGD2, the dominant mast cells derived prostanoid species, has been added to the growing list of Th2 effectors. Prostaglandin D2 synthase (hPGDS) is expressed in human Th2 but not Th1 cells. The upregulated expression of hPGDS and de novo expression of COX-2, induced with anti-CD3, result in the production of PGD2 by Th2 cells (Tanaka, K. et al. (2000) J. Immunol. 164:2277-2280). Of note, mice rendered deficient in the PGD2 receptor have impaired Th2 responses and allergen-induced airway hyperresponsiveness in a model of allergic asthma (Matsuoka, T. et al. (2000) Science 287:2013-2017).

### SUMMARY OF THE INVENTION

The present invention reports the molecular cloning of a novel PLA₂, designated group XII PLA₂ (GXII PLA₂), which contains at least two alternatively spliced forms, GXII-1 and GXII-2. Both forms of this PLA₂, GXII-1 (GXII-1) PLA₂ and GrXII-2 (GXII-2) PLA₂, contain a histidine based PLA₂ catalytic domain and are cysteine-rich although the position and spacing of the cysteine residues are different from those in other PLA₂s. Recombinant GXII PLA₂ is enzymatically active and prefers arachidonic acid as a substrate in the sn-2 position of phosphotidylethanolamine, whereas other cysteine-rich groups are not selective. Importantly, GXII PLA₂, in particular GXII-2 PLA₂, is preferentially expressed in Th2 cells where it is induced upon stimulation through the T cell receptor. Thus, GXII PLA₂ is novel by its 20 kDa size, distribution of cysteine residues, and preference of arachidonic acid in comparison to other cysteine-rich, catalytic histidine based PLA₂ groups, and by its preferential expression in Th2 cells. Additionally, it has now been demonstrated that another PLA₂, GV PLA₂, also is preferentially expressed in Th2 cells.

This invention pertains to isolated compositions of GXII PLA₂ protein and isolated nucleic acid sequences encoding GXII PLA₂, other compositions related thereto and methods of use thereof. The amino acid sequence of a human GXII PLA₂ protein, referred to as hGXII-1 PLA₂, has been determined (shown in SEQ ID NO: 2) and a CDNA encoding this human GXII-1 PLA₂ protein has been isolated (the nucleotide sequence of which is shown in SEQ ID NO: 1). The amino acid sequence of a mouse GXII PLA₂ protein, referred to as mGXII-1 PLA₂, also has been determined (shown in SEQ ID NO: 4) and a cDNA encoding this mouse GXII-1 PLA₂ protein has been isolated (the nucleotide sequence of which is shown in SEQ ID NO: 3). The amino acid sequence of another mouse GXII PLA₂ protein, referred to as mGXII-2 PLA₂, also has been determined (shown in SEQ ID NO: 6) and a cDNA encoding this mouse GXII-2 PLA₂ protein has been isolated (the nucleotide sequence of which is shown in SEQ ID NO: 5). The mGXII-2 PLA₂ form represents an alternatively spliced form of mGXII-1 PLA₂.

In one aspect, the invention pertains to isolated nucleic acid molecules comprising a nucleotide sequence encoding GXII PLA₂, or a biologically active portion thereof. In one embodiment, the invention provides an isolated nucleic acid molecule comprising a nucleotide sequence encoding a protein, wherein: (i) the protein comprises an amino acid sequence at least 60% homologous to the amino acid sequence of SEQ ID NO: 2, SEQ ID NO: 4 or SEQ ID NO: 6, or (ii) a nucleotide sequence is at least 60% homologous to the nucleotide sequence of SEQ ID NO: 1, SEQ ID NO: 3 or SEQ ID NO: 5; and wherein the protein selectively hydrolyzes arachidonic acid in the sn-2 position of phosphatidylethanolamine. Alternatively, the protein can comprise an amino acid sequence at least 70%, 80%, 90% or more homologous to the amino acid sequence of SEQ ID NO: 2, SEQ ID NO: 4 or SEQ ID NO: 6 or the nucleotide sequence can be at least 70%, 80%, 90% or more homologous to the nucleotide sequence of SEQ ID NO: 1, SEQ ID NO: 3 or SEQ ID NO: 5. In another embodiment, the invention provides an isolated nucleic acid molecule at least 30 nucleotides in length which hybridizes under stringent conditions to the nucleic acid molecule of SEQ ID NO: 1, SEQ ID NO: 3 or SEQ ID NO: 5. Alternatively, the nucleic acid molecule can be at least 50, 100, 300, 500 or 700 nucleotides in length. The nucleic acid molecule can comprise a naturally-occurring nucleotide sequence, such as a naturally-occurring mouse GXII PLA₂ or human GXII PLA₂. In another embodiment, the invention provides an isolated nucleic acid molecule comprising the coding region of the nucleotide sequence of SEQ ID NO: 1, SEQ ID NO: 3 or SEQ ID NO: 5. In another embodiment, the invention provides an isolated nucleic acid molecule of claim 15, comprising the nucleotide sequence of SEQ ID NO: 1, SEQ ID NO: 3 or SEQ ID NO: 5. In yet another embodiment, the invention provides an isolated nucleic acid molecule encoding the amino acid sequence of SEQ ID NO: 2, SEQ ID NO: 4 or SEQ ID NO: 6. Isolated nucleic acid molecules encoding GXII PLA₂ fusion proteins and isolated nucleic acid molecules that are antisense to a nucleic acid molecule encoding GXII PLA₂ are also provided.

Another aspect of the invention pertains to vectors, such as recombinant expression vectors, containing an nucleic acid molecule of the invention and host cells into which such vectors have been introduced. In one embodiment, such a host cell is used to produce GXII PLA₂ protein by culturing the host cell in a suitable medium. If desired, GXII PLA₂ protein can be then isolated from the host cell or the medium.

Still another aspect of the invention pertains to isolated GXII PLA₂ proteins, or biologically active portions thereof. In one embodiment, the invention provides an isolated protein which comprises an amino acid sequence homologous to the amino acid sequence of SEQ ID NO: 2, SEQ ID NO: 4 or SEQ ID NO: 6, wherein the protein selectively hydrolyzes arachidonic acid in the sn-2 position of phosphatidylethanolamine. Fusion proteins, comprising a GXII PLA₂ polypeptide linked to a non-GXII PLA₂ polypeptide, are also provided. Antigenic peptides of GXII PLA₂ are also provided.

The GXII PLA₂ proteins of the invention, or fragments thereof, can be used to prepare anti-GXII PLA₂ antibodies. Accordingly, the invention further provides an antibody that specifically binds GXII PLA₂ protein. In one embodiment, the antibody is monoclonal. In another embodiment, the antibody is labeled with a detectable substance.

The GXII PLA₂-encoding nucleic acid molecules of the invention can be used to prepare nonhuman transgenic animals that contain cells carrying a transgene encoding GXII PLA₂ protein or a portion of GXII PLA₂ protein. Accordingly, such transgenic animals are also provided by the invention. In one embodiment, the GXII PLA₂ transgene carried by the transgenic animal alters an endogenous gene encoding endogenous GXII PLA₂ protein (e.g., a homologous recombinant animal).

Another aspect of the invention pertains to methods for detecting the presence of GXII PLA₂ protein or mRNA in a biological sample. To detect GXII PLA₂ protein or mRNA, the biological sample is contacted with an agent capable of detecting GXII PLA₂ protein (such as a labeled anti-GXII PLA₂ antibody) or GXII PLA₂ mRNA (such as a labeled nucleic acid probe capable of hybridizing to GXII PLA₂ mRNA) such that the presence of GXII PLA₂ protein or mRNA is detected in the biological sample.

Still another aspect of the invention pertains to methods for identifying compounds that modulate the activity or expression of GXII PLA₂, wherein an indicator composition comprising GXII PLA₂ and a substrate for GXII PLA₂ is contacted with a test compound and the effect of the test compound on GXII PLA₂ activity toward the substrate in the indicator composition is determined. Preferably, the substrate is arachidonic acid in the sn-2 position of phosphatidylethanolamine.

Still another aspect of the invention pertains to modulatory methods. In one embodiment, the invention provides a method for modulating Th2 cell activity comprising contacting Th2 cells, or precursors thereof, with a modulator of GXII PLA₂ or GV PLA₂ such thatTh2 cell activity is modulated. In another embodiment, the invention provides a method for modulating prostaglandin production by Th2 cells comprising contacting Th2 cells with a modulator of GXII PLA₂ or GV PLA₂ such that prostaglandin production by the Th2 cells is modulated.

### BRIEF DESCRIPTION OF THE DRAWINGS

FIG. 1 is a comparison of the amino acid sequences of GXII PLA₂s and other low molecular weight PLA₂s. All sequences listed are in single letter amino acid code. The numbers on the right hand side are the numbers of the amino acid residues of each low molecular weight PLA₂. The catalytic domain, the conserved cysteine residues, and the calcium binding domain of low molecular weight PLA₂s are boxed with thick, thin, and dotted lines, respectively. The cysteine residues that are conserved among low molecular weight PLA₂ and are used to form disulfide bonds are also numbered, according to Valentin, E. et al. (1999) J. Biol. Chem. 274:31195-31202, in the bottom of boxes. All the sequences listed are mouse protein sequences (m) with the exception of the human GXII-1 PLA₂ (hGXII-1). The alignment was created by using Lasergene program (DNASTAR). The sequences are also disclosed in the Sequence Listing as follows: mGXII-1-SEQ ID NO: 4; hGXII-1-SEQ ID NO: 2; mGXII-2-SEQ ID NO: 6; mGI-SEQ ID NO: 7; mGIIA-SEQ ID NO: 8; mGV-SEQ ID NO: 9; mGX-SEQ ID NO: 10.

FIG. 2A is an SDS-PAGE analysis of recombinant mGXII-1 PLA₂. Control vector or mGXII-1 PLA₂ vector transformed E. coli strain was left uninduced (−) or induced with IPTG (+) for 3 hours. The whole bacterial extracts were fractionated on a 12% SDS-polyacrylamide gel and stained with Coomassie brilliant blue.

FIGS. 2B-2E are graphs depicting the effect of Ca2+ concentration (FIG. 2B), pH (FIG. 2C), DTT concentration (FIG. 2D) and different substrates (FIG. 2E) on the enzymatic activity of mGXII-1 PLA₂. Ca²⁺-dependence (FIG. 2B), pH-dependence (FIG. 2C), inhibition by DTT (FIG. 2D), and substrate specificity (FIG. 2E, solid bars) were determined as described in the section on Materials and Methods below. The substrate specificity of GIB PLA₂, from procine pancreas (FIG. E, open bars), was also determined for comparison. PL-PE, PA-PE, PA-PC, PP-PC, SA-PC, and SA-PI stand for 1-palmitoyl-2-[¹⁴C]linoleoyl-phophatidylethanolamine, 1-palmitoyl-2-[¹⁴C]arachidonoyl-phosphatidylethanolamine, 1-palmitoyl-2-[¹⁴C]arachidonoyl-phophatidylcholine, 1-palmatoyl-2-[¹⁴C]palmitoyl-phosphotidylcholine, 1-stearoyl-2-[¹⁴C]arachidonoyl-phosphatidylcholine, and 1-stearoyl-2-[¹⁴C]arachidonoyl-phophotidylinositol, respectively.

FIGS. 3A-B depict expression of mGXII PLA₂ transcripts in murine Th1 and Th2 cells. 10 μg of each indicated total RNA sample was fractionated on 1.2% formaldehyde agarose gels, transferred to nitrocellulose membranes and hybridized with the indicated cDNA probes. In FIG. 3A, total RNA was derived from either resting, ionomycin (1 μM, Ion) stimulated, or anti-CD3 stimulated Th1 clones (D1.1, OF6, and AR5) or Th2 clones (D10 and CDC35). In FIG. 3B, naïve CD4+Th cells were differentiated in vitro into either Th1 or Th2 cells as described in the Materials and Methods section below. At the indicated time points, total RNA was prepared and subjected to Northern analysis. “Hr” and “d” stand for hours and days after stimulation, respectively.

FIGS. 4A-4B depict RT-PCR analysis of mGXII-1, mGXII-2, mGV, and mGX PLA₂s. 0.5 μg of total RNA prepared from in vitro differentiated Th1 and Th2 cells (either resting or 6 hours after anti-CD3 stimulation), and purified primary B cells (stimulated with PMA/Ionomycin for 6 hours) (FIG. 4A), and from various organs (FIG. 4B) was used in RT-PCR reactions as described in the Materials and Methods section below. β-actin was also amplified as a control for the amount of input RNA.

FIG. 4C depicts Northern blot analysis of mGV PLA₂ in Th clones. Total RNA was prepared from resting or anti-CD3 stimulated D10 and AE7 cells and subjected to Northern analysis for the expression of mGV PLA₂. The same blot was also hybridized with a γ-actin cDNA probe as a control for the amount of input RNA.

FIG. 5 is a bar graph depicting decreased anti-CD3-induced proliferation of splenocytes derived from GXII PLA₂ heterozygous mice. Splenocytes prepared from GXII PLA₂ heterozygous mice (Het) or wild type littermates (WT) were plated at 500,000 cells/100 μl medium/well and stimulated with plate-bound anti-CD3, at indicated concentrations, for 48 hours prior to the addition of ³H-thymidine (1 μCu/50 μl medium/well). Sixteen hours after the addition of ³H-thymidine, cells were harvested and the uptake of ³H-thymidine was measured by a scintillation counter. This figure represents two independent experiments.

FIG. 6 is a bar graph depicting decreased production of IL-4 by GXII PLA₂ heterozygous Th cells. CD4+Th cells were purified from GXII PLA₂ heterozygous mice (Het) or wild type littermates (WT) and differentiated in vitro under the non-skewing (NS), Th1-skewing (Th1), or Th2-skewing (Th2) condition. Seven days after primary stimulation, cells were replated at 500,000 cell/ml and stimulated with plate-bound anti-CD3. Twenty-four hours after secondary stimulation, supernatants were harvested and the IL-4 concentration was measured by ELISA. This figure represents two independent experiments.

FIG. 7 is a schematic diagram of the wild type mGXII PLA₂ gene and its mutant allele created by homologous recombination with a “knockout” construct. The filled boxes represent the exons of the mGXII PLA₂ gene (not all exons are included in this diagram). A, B, Bgl, C, E and X represent AccI, BamHI, BglII, ClaI, EcoRV and XhoI restriction sites, respectively. Neo represents the neomycin gene. The arrow indicates the 5′ to 3′ orientation of the neomycin gene.

### DETAILED DESCRIPTION OF THE INVENTION

This invention describes the molecular cloning and characterization of a novel PLA₂ group, designated group XII. While this novel PLA₂ group possesses the generic PLA₂ characteristics of being cysteine-rich, DTT-sensitive, and flnctioning optimally at a basic pH, GXII PLA₂ merits this separate designation because of the following features. It shares no significant amino acid sequence homology, other than the catalytic histidine motif, with other PLA₂ enzymes. Although GXII PLA₂ is cysteine-rich, the spacing of cysteine residues is distinct from that of other low molecular weight PLA₂ enzymes. GXII PLA₂ also lacks the canonical calcium-binding domain of the low molecular weight PLA₂ enzymes. The molecular weight of GXII-1 PLA₂ is approximately 20 kDa, which is somewhat larger than the 14 kDa of the low molecular weight cysteine-rich PLA₂ enzymes. In addition, GXII PLA₂ is present in more than one species and alternative spliced forms (mGXII-1 and mGXII-2) have been identified. An identified alternatively spliced form, GXII-2 PLA₂, does not contain any signal peptide. Most low molecular weight PLA₂ members are either secreted proteins or are associated with cell membranes and act in situ. GXII-2 PLA₂, without a signal peptide, may be the only exception within the low molecular weight PLA₂ family. Importantly, whilst the family of low molecular weight enzymes have little substrate specificity, GXII-1 PLA₂ has a preference for arachidonic acid in the sn-2 position of phosphatidylethanolamine. GXII PLA₂, in particular GXII-2 PLA₂, and GV PLA₂ are preferentially expressed in Th2 cells within the T cell lineage.

So that the invention may be more readily understood, certain terms are first defined.

As used herein the term “phospholipase A₂” (abbreviated as PLA₂) refers to any member of a family of enzymes characterized by having the enzymatic activity of hydrolyzing the sn-2 ester bond of phospholipids to release free fatty acids.

As used herein, the term “GXII PLA₂” is intended to encompass splice variants, including both long (GXII-1 PLA₂) and short (GXII-2 PLA₂) forms of GXII PLA₂.

As used herein, the term “nucleic acid molecule” is intended to include DNA molecules (e.g., cDNA, genomic DNA, oligonucleotides) and RNA molecules (e.g., mRNA). The nucleic acid molecule may be single-stranded or double-stranded, but preferably is double-stranded DNA.

As used herein, the term “isolated” or “purified” nucleic acid molecule includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. A preferred, example of stringent hybridization conditions are hybridization in 6× sodium chloride/sodium citrate (SSC) at about 45° C., followed by one or more washes in 0.2× SSC, 0.1% SDS at 50° C. Another example of stringent hybridization conditions are hybridization in 6× sodium chloride/sodium citrate (SSC) at about 45° C., followed by one or more washes in 0.2× SSC, 0.1% SDS at 55° C. A further example of stringent hybridization conditions are hybridization in 6× sodium chloride/sodium citrate (SSC) at about 45° C., followed by one or more washes in 0.2× SSC, 0.1% SDS at 60° C. Particularly preferred stringency conditions (and the conditions that should be used if the practitioner is uncertain about what conditions should be applied to determine if a molecule is within a hybridization limitation of the invention) are hybridization in 6× sodium chloride/sodium citrate (SSC) at about 45° C., followed by one or more washes in 0.2× SSC, 0.1% SDS at 65° C. Preferably, an isolated nucleic acid molecule of the invention that hybridizes under stringent conditions to the sequence of SEQ ID NO:1, SEQ ID NO:3 or SEQ ID NO:5, corresponds to a naturally-occurring nucleic acid molecule.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of a GXII PLA₂ protein having less than about 30%, 20%, 10% and more preferably 5% (by dry weight), of non-GXII PLA₂ protein (also referred to herein as a “contaminating protein”), or of chemical precursors or non-GXII PLA₂ chemicals. When the GXII PLA₂ protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

As used herein, a “naturally-occurring” nucleic acid molecule refers to an RNA or DNA molecule having a nucleotide sequence that occurs in nature (e.g., encodes a natural protein).

As used herein, an “antisense” nucleic acid comprises a nucleotide sequence which is complementary to a “sense” nucleic acid encoding a protein, e.g., complementary to the coding strand of a double-stranded cDNA molecule, complementary to an MRNA sequence or complementary to the coding strand of a gene. Accordingly, an antisense nucleic acid can hydrogen bond to a sense nucleic acid.

As used herein, the term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, the term “host cell” is intended to refer to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, a “transgenic animal” refers to a non-human animal, preferably a mammal, more preferably a mouse, in which one or more of the cells of the animal includes a “transgene”. The term “transgene” refers to exogenous DNA which is integrated into the genome of a cell from which a transgenic animal develops and which remains in the genome of the mature animal, for example directing the expression of an encoded gene product in one or more cell types or tissues of the transgenic animal, or disrupting the normal expression of the endogenous gene into which the exogenous DNA has been inserted.

As used herein, a “homologous recombinant animal” refers to a type of transgenic non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

As used herein, the term “antibody” is intended to include immunoglobulin molecules and immunologically active portions of immunoglobulin molecules, i.e., molecules that contain an antigen binding site which specifically binds (immunoreacts with) an antigen, such as Fab and F(ab′).sub.2 fragments. The terms “monoclonal antibody” and “monoclonal antibody composition”, as used herein, refer to a population of antibody molecules that contain only one species of an antigen binding site capable of immunoreacting with a particular epitope of an antigen. A monoclonal antibody composition thus typically displays a single binding affinity for a particular antigen with which it immunoreacts.

Various aspects of the invention are described in further detail in the following subsections:

I. Isolated Nucleic Acid Molecules

One aspect of the invention pertains to isolated nucleic acid molecules that encode GXII PLA₂, or biologically active fragments thereof. In a preferred embodiment, an isolated nucleic acid molecule of the invention comprises the nucleotide sequence shown in SEQ ID NO: 1, SEQ ID NO: 3 or SEQ ID NO: 5. The sequence of SEQ ID NO: 1 corresponds to a human GXII-1 PLA₂ cDNA. This cDNA comprises sequences encoding the hGXII-1 PLA₂ protein (i.e., “the coding region”, from nucleotides 37-603), as well as 5′ untranslated sequences (nucleotides 1-36) and 3′ untranslated sequences (nucleotides 604-1044). Alternatively, the nucleic acid molecule may comprise only the coding region of SEQ ID NO: 1 (i.e., nucleotides 37-603). The sequence of SEQ ID NO: 3 corresponds to a mouse GXII-1 PLA₂ cDNA. This cDNA comprises sequences encoding the mGXII-1 PLA₂ protein (i.e., “the coding region”, from nucleotides 87-662), as well as 5′ untranslated sequences (nucleotides 1-86) and 3′ untranslated sequences (nucleotides 663-1529). Alternatively, the nucleic acid molecule may comprise only the coding region of SEQ ID NO: 3 (i.e., nucleotides 87-662). The sequence of SEQ ID NO: 5 corresponds to a mouse GXII-2 PLA₂ cDNA. This cDNA comprises sequences encoding the mGXII-2 PLA₂ protein (i.e., “the coding region”, from nucleotides 14-472), as well as 5′ untranslated sequences (nucleotides 1-13) and 3′ untranslated sequences (nucleotides 473-476). Alternatively, the nucleic acid molecule may comprise only the coding region of SEQ ID NO: 5 (i.e., nucleotides 14-472).

Moreover, the nucleic acid molecule of the invention can comprise only a portion of the coding region of SEQ ID NO: 1, SEQ ID NO:3 or SEQ IDNO:5, for example a fragment encoding a biologically active portion of GXII PLA₂. The term “biologically active portion of GXII PLA₂” is intended to include portions of GXII PLA₂ that retain enzymatic activity, e.g., the ability to hydrolyze arachidonic acid in the sn-2 position of phosphatidylethanolamine. A region comprising the amino acid sequence CCNQHDRCY (amino acid positions 106-114 of SEQ ID NO:2, positions 109-117 of SEQ ID NO:4 and positions 70-78 of SEQ ID NO:6) has been identified as being necessary for maintenance of enzymatic activity, and thus should be maintained in the biologically active portion. The ability of portions of GXII PLA₂ to hydrolyze a substrate (e.g., arachidonic acid) can be determined in standard in vitro hydrolysis assays, for example using a recombinant GXII PLA₂ protein and the liposome based assay described in the Examples. Nucleic acid fragments encoding biologically active portions of GXII PLA₂ can be prepared by isolating a portion of SEQ ID NO: 1, SEQ ID NO:3 or SEQ ID NO: 5, expressing the encoded portion of GXII PLA₂ protein or peptide (e.g., by recombinant expression in a host cell) and assessing the ability of the portion hydrolyze an appropriate substrate (e.g., arachidonic acid in the sn-2 position of phosphatidylenthanolamine) using an assay such as that described in the Examples.

The invention further encompasses nucleic acid molecules that differ from SEQ ID NO: 1, SEQ ID NO:3 or SEQ ID NO:5 (and fragments thereof) due to degeneracy of the genetic code and thus encode the same GXII PLA₂ proteis as those encoded by SEQ ID NO: 1, SEQ ID NO:3 or SEQ ID NO: 5. Accordingly, in another embodiment, an isolated nucleic acid molecule of the invention has a nucleotide sequence encoding a protein having an amino acid sequence shown in SEQ ID NO: 2, SEQ ID NO:4 or SEQ ID NO:6. Moreover, the invention encompasses nucleic acid molecules that encode portions of SEQ ID NO: 2, SEQ ID NO: 4 or SEQ ID NO:6, such as biologically active portions thereof.

A nucleic acid molecule having the nucleotide sequence of SEQ ID NO: 1, SEQ ID NO:3 or SEQ ID NO:5, or a portion thereof, can be isolated using standard molecular biology techniques and the sequence information provided herein. For example, a GXII PLA₂ cDNA can be isolated from a cDNA library using all or portion of SEQ ID NO: 1, SEQ ID NO:3 or SEQ ID NO:5 as a hybridization probe and standard hybridization techniques (e.g., as described in Sambrook, J., et al. Molecular Cloning: A Laboratory Manual. 2nd, ed., Cold Spring Harbor Laboratory, Cold Spring Harbor, N.Y., 1989). Moreover, a nucleic acid molecule encompassing all or a portion of SEQ ID NO: 1, SEQ ID NO:3 or SEQ ID NO:5 can be isolated by the polymerase chain reaction using oligonucleotide primers designed based upon the sequence of SEQ ID NO: 1, SEQ ID NO: 3 or SEQ ID NO:5. For example, mRNA can be isolated from cells (e.g., by the guanidinium-thiocyanate extraction procedure of Chirgwin et al. (1979) Biochemistry 18: 5294-5299) and cDNA can be prepared using reverse transcriptase (e.g., Moloney MLV reverse transcriptase, available from Gibco/BRL, Bethesda, Md.; or AMC reverse transcriptase, available from Seikagaku America, Inc., St. Petersburg, Fla.). Synthetic oligonucleotide primers for PCR amplification can be designed based upon the nucleotide sequence shown in SEQ ID NO: 1, SEQ ID NO:3 or SEQ ID NO:5. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to a GXII PLA₂ nucleotide sequence can be prepared by standard synthetic techniques, e.g., using an automated DNA synthesizer.

In addition to the GXII PLA₂ nucleotide sequence shown in SEQ ID NO: 1, SEQ ID NO:3 and SEQ ID NO:5, it will be appreciated by those skilled in the art that DNA sequence polymorphisms that lead to changes in the amino acid sequences of GXII PLA₂ may exist within a population. Such genetic polymorphism in the GXII PLA₂ gene may exist among individuals within a population due to natural allelic variation. Such natural allelic variations can typically result in 1-5% variance in the nucleotide sequence of the a gene. Any and all such nucleotide variations and resulting amino acid polymorphisms in GXII PLA₂ that are the result of natural allelic variation and that do not alter the functional activity of GXII PLA₂ are intended to be within the scope of the invention. Moreover, nucleic acid molecules encoding GXII PLA₂ proteins from other species, and thus which have a nucleotide sequence that differs from the human and mouse sequences of SEQ ID NO: 1, SEQ ID NO:3 and SEQ ID NO:5, but that are related to the human and mouse sequences, are intended to be within the scope of the invention. Nucleic acid molecules corresponding to natural allelic variants and other mammalian homologues of the human and mouse GXII PLA₂ cDNAs of the invention can be isolated based on their homology to the human and/or mouse GXII PLA₂ nucleic acid molecule disclosed herein using a human or mouse cDNA, or a portion thereof, as a hybridization probe according to standard hybridization techniques under stringent hybridization conditions. Accordingly, in another embodiment, an isolated nucleic acid molecule of the invention hybridizes under stringent conditions to the nucleic acid molecule comprising the nucleotide sequence of SEQ ID NO: 1, SEQ ID NO: 3 or SEQ ID NO: 5. In certain embodiment, the nucleic acid is at least 15, 30, 50, 100, 200, 300, 400, 500, 600, 700, 800, 900, 1000 or 1500 nucleotides in length. Preferably, an isolated nucleic acid molecule of the invention that hybridizes under stringent conditions to the sequence of SEQ ID NO: 1, SEQ ID NO: 3 or SEQ ID NO: 5 corresponds to a naturally-occurring nucleic acid molecule. In on embodiment, the nucleic acid encodes a natural human GXII PLA₂ protein. In another embodiment, the nucleic acid molecule encodes a natural murine GXII PLA₂ protein, such as mouse GXII PLA₂ protein.

In addition to naturally-occurring allelic variants of the GXII PLA₂ sequence that may exist in the population, the skilled artisan will further appreciate that changes may be introduced by mutation into the nucleotide sequence of SEQ ID NO: 1, SEQ ID NO: 3 or SEQ ID NO:5 thereby leading to changes in the amino acid sequence of the encoded protein, without altering the functional activity of the GXII PLA₂ protein. For example, nucleotide substitutions leading to amino acid substitutions at “non-essential” amino acid residues may be made in the sequence of SEQ ID NO: 1, SEQ ID NO:3 or SEQ ID NO:5. A “non-essential” amino acid residue is a residue that can be altered from the wild-type sequence of GXII PLA₂ (e.g., the sequence of SEQ ID NO: 2, SEQ ID NO:4 or SEQ ID NO:6) without altering the functional activity of GXII PLA₂, such as its ability to hydrolyze arachidonic acid in the sn-2 position of phosphatidylethanolamine, whereas an “essential” amino acid residue is required for functional activity. A region comprising the amino acid sequence CCNQHDRCY (amino acid positions 106-114 of SEQ ID NO:2, positions 109-117 of SEQ ID NO:4 and positions 70-78 of SEQ ID NO:6) has been identified as being necessary for maintenance of enzymatic activity, and thus these amino acid residues should be maintained as essential amino acid residues.

Accordingly, another aspect of the invention pertains to nucleic acid molecules encoding GXII PLA₂ proteins that contain changes in amino acid residues that are not essential for GXII PLA₂ activity. Such GXII PLA₂ proteins differ in amino acid sequence from SEQ ID NO: 2, SEQ ID NO:4 or SEQ ID NO:6 yet retain GXII PLA₂ activity. In one embodiment, the isolated nucleic acid molecule comprises a nucleotide sequence encoding a protein, wherein the protein comprises an amino acid sequence at least 60% homologous to the amino acid sequence of SEQ ID NO: 2, SEQ ID NO:4 or SEQ ID NO:6 and wherein the protein selectively hydrolyzes arachidonic acid in the sn-2 position of phosphatidylethanolamine. “Selectively hydrolyzes arachidonic acid in the sn-2 position of phosphatidylethanolamine” is intended to mean that the protein demonstrates a preference for this substrate as compared to other substrates, as described further in Example 3. Preferably, the protein encoded by the nucleic acid molecule is at least 70% homologous to SEQ ID NO: 2, SEQ ID NO: 4 or SEQ ID NO: 6, more preferably at least 80% homologous to SEQ ID NO: 2, SEQ ID NO:4 or SEQ ID NO:6, even more preferably at least 90% homologous to SEQ ID NO: 2, SEQ ID NO:4 or SEQ ID NO:6 and most preferably at least 95%, 96%, 97%, 98%, 99% or 99.5% homologous to SEQ ID NO: 2, SEQ ID NO:4 or SEQ ID NO:6.

To determine the percent homology of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence (e.g., when aligning a second sequence to the hGXII-1 PLA₂ amino acid sequence of SEQ ID NO:2 having 189 amino acid residues, at least 57, preferably at least 75, more preferably at least 95, even more preferably at least 113, and even more preferably at least 132, 151, 170, or 189 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) is using a Blossum 62 scoring matrix with a gap open penalty of 12, a gap extend penalty of 4, and a frameshift gap penalty of 5.

The percent identity between two amino acid or nucleotide sequences can be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990) J. Mol. Biol. 215:403-10. BLAST nucleotide searches can be performed with the NBLAST program, score=100, wordlength=12 to obtain nucleotide sequences homologous to 57242 nucleic acid molecules of the invention. BLAST protein searches can be performed with the XBLAST program, score=50, wordlength=3 to obtain amino acid sequences homologous to 57242 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

An isolated nucleic acid molecule encoding a GXII PLA₂ protein homologous to the protein of SEQ ID NO: 2, SEQ ID NO: 4 or SEQ ID NO:6 can be created by introducing one or more nucleotide substitutions, additions or deletions into the nucleotide sequence of SEQ ID NO: 1, SEQ ID NO:3 or SEQ ID NO:5 such that one or more amino acid substitutions, additions or deletions are introduced into the encoded protein. Mutations can be introduced into SEQ ID NO: 1, SEQ ID NO:3 or SEQ ID NO:5 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, an amino acid residue in GXII PLA₂ protein is preferably replaced with another amino acid residue from the same side chain family. Alternatively, in another embodiment, mutations can be introduced randomly along all or part of a GXII PLA₂ coding sequence, such as by saturation mutagenesis, and the resultant mutants can be screened for their ability to hydrolyze an appropriate subststrate (e.g, arachidonic acid in the sn-2 position of phosphatidylethanolamine) to identify mutants that retain enzymatic activity. Following mutagenesis of SEQ ID NO: 1, SEQ ID NO:3 or SEQ ID NO:5, the encoded mutant protein can be expressed recombinantly in a host cell and the enzymatic activity of the mutant protein can be determined using an in vitro assay for phospholipase A₂ activity as described in Example 3.

Another aspect of the invention pertains to isolated nucleic acid molecules that are antisense to the coding strand of a GXII PLA₂ mRNA or gene. An antisense nucleic acid of the invention can be complementary to an entire GXII PLA₂ coding strand, or to only a portion thereof. In one embodiment, an antisense nucleic acid molecule is antisense to a coding region of the coding strand of a nucleotide sequence encoding GXII PLA₂ (e.g., the entire coding region of SEQ ID NO: 1 comprises nucleotides 37-603, the entire coding region of SEQ ID NO:3 comprises nucleotides 87-662 and the entire coding region of SEQ ID NO:5 comprises nucleotides 14-472). In another embodiment, the antisense nucleic acid molecule is antisense to a noncoding region of the coding strand of a nucleotide sequence encoding GXII PLA₂. In certain embodiments, an antisense nucleic acid of the invention is at least 15, 30, 50, 100, 200, 300,400, 500,600, 700, 800, 900, 1000 or 1500 nucleotides in length.

Given the coding strand sequences encoding GXII PLA₂ disclosed herein (e.g., SEQ ID NO: 1, SEQ ID NO:3 or SEQ ID NO:5), antisense nucleic acids of the invention can be designed according to the rules of Watson and Crick base pairing. The antisense nucleic acid molecule may be complementary to the entire coding region of GXII PLA₂ mRNA, or alternatively can be an oligonucleotide which is antisense to only a portion of the coding or noncoding region of GXII PLA₂ mRNA. For example, the antisense oligonucleotide may be complementary to the region surrounding the translation start site of GXII PLA₂ mRNA. An antisense oligonucleotide can be, for example, about 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. A ribozyme having specificity for a GXII PLA₂-encoding nucleic acid can be designed based upon the nucleotide sequence of a GXII PLA₂ cDNA disclosed herein (i.e., SEQ ID NO: 1, SEQ ID NO:3 or SEQ ID NO:5). For example, a derivative of a Tetrahymena L-19 IVS RNA can be constructed in which the base sequence of the active site is complementary to the base sequence to be cleaved in a GXII PLA₂-encoding MRNA. See for example Cech et al. U.S. Pat. No. 4,987,071; and Cech et al. U.S. Pat. No. 5,116,742. Alternatively, GXII PLA₂ mRNA can be used to select a catalytic RNA having a specific ribonuclease activity from a pool of RNA molecules. See for example Bartel, D. and Szostak, J. W. (1993) Science 261: 1411-1418.

Yet another aspect of the invention pertains to isolated nucleic acid molecules encoding GXII PLA₂ fusion proteins. Such nucleic acid molecules, comprising at least a first nucleotide sequence encoding a GXII PLA₂ protein, polypeptide or peptide operatively linked to a second nucleotide sequence encoding a non-GXII PLA₂ protein, polypeptide or peptide, can be prepared by standard recombinant DNA techniques. GXII PLA₂ fusion proteins are described in further detail below in subsection III.

II. Recombinant Expression Vectors and Host Cells

Another aspect of the invention pertains to vectors, preferably recombinant expression vectors, containing a nucleic acid encoding GXII PLA₂ (or a portion thereof). The expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals) well known in the art. Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector may depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., GXII PLA₂ proteins, mutant forms of GXII PLA₂ proteins, GXII PLA₂ fusion proteins and the like).

The recombinant expression vectors of the invention can be designed for expression of GXII PLA₂ protein in prokaryotic or eukaryotic cells. For example, GXII PLA₂ can be expressed in bacterial cells such as E. coli, insect cells (using baculovirus expression vectors), yeast cells or mammalian cells. Suitable host cells are well known in the art. Alternatively, the recombinant expression vector may be transcribed and translated in vitro, for example using T7 promoter regulatory sequences and T7 polymerase.

Expression of proteins in prokaryotes is most often carried out in E. coli (e.g., BL21 E. coli cells as described in the Materials and Methods section below) with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc.), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc and pET 11d. Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident .lambda. prophage harboring a T7 gn1 gene under the transcriptional control of the lacUV 5 promoter.

One strategy known in the art to maximize recombinant protein expression in E. coli is to express the protein in a host bacteria with an impaired capacity to proteolytically cleave the recombinant protein. Another strategy is to alter the nucleic acid sequence of the nucleic acid to be inserted into an expression vector so that the individual codons for each amino acid are those preferentially utilized in E. coli. Such alteration of nucleic acid sequences of the invention can be carried out by standard DNA synthesis techniques.

In another embodiment, the GXII PLA₂ expression vector is a yeast expression vector. Examples of vectors for expression in yeast S. cerivisae include pYepSecl (Baldari.et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.).

Alternatively, GXII PLA₂ can be expressed in insect cells using baculovirus expression vectors. Baculovirus vectors available for expression of proteins in cultured insect cells (e.g., Sf9 cells) include the pAc series (Smith et al., (1983) Mol. Cell Biol. 3:2156-2165) and the pVL series (Lucklow, V. A., and Summers, M. D., (1989) Virology 170:31-39).

In yet another embodiment, a nucleic acid of the invention is expressed in mammalian cells using a mammalian expression vector. Examples of mammalian expression vectors include pMex-NeoI, pCDM8 (Seed, B., (1987) Nature 329:840) and pMT2PC (Kaufman et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), the albumin promoter (liver-specific; Pinkert et al. (1987) Genes Dev. 1:268-277), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the alpha-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to GXII PLA₂mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to recombinant host cells into which a vector, preferably a recombinant expression vector, of the invention has been introduced. A host cell may be any prokaryotic or eukaryotic cell. For example, GXII PLA₂ protein may be expressed in bacterial cells such as E. coli, insect cells, yeast or mammalian cells (such as Chinese hamster ovary cells (CHO) or COS cells). Other suitable host cells are known to those skilled in the art. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells are well known in the art.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker may be introduced into a host cell on the same vector as that encoding GXII PLA₂ or may be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) GXII PLA₂ protein. Accordingly, the invention further provides methods for producing GXII PLA₂ protein using the host cells of the invention. In one embodiment, the method comprises culturing the host cell of invention (into which a recombinant expression vector encoding GXII PLA₂ has been introduced) in a suitable medium until GXII PLA₂ is produced. In another embodiment, the method further comprises isolating GXII PLA₂ from the medium or the host cell.

Certain host cells of the invention can also be used to produce nonhuman transgenic animals. For example, in one embodiment, a host cell of the invention is a fertilized oocyte or an embryonic stem cell into which GXII PLA₂-coding sequences have been introduced. Such host cells can then be used to create non-human transgenic animals in which exogenous GXII PLA₂ sequences have been introduced into their genome or homologous recombinant animals in which endogenous GXII PLA₂ sequences have been altered. Such animals are useful for studying the function and/or activity of GXII PLA₂ and for identifying and/or evaluating modulators of GXII PLA₂ activity. Accordingly, another aspect of the invention pertains to nonhuman transgenic animals which contain cells carrying a transgene encoding a GXII PLA₂ protein or a portion of a GXII PLA₂ protein. In a subembodiment, of the transgenic animals of the invention, the transgene alters an endogenous gene encoding an endogenous GXII PLA₂ protein (e.g., homologous recombinant animals in which the endogenous GXII PLA₂ gene has been functionally disrupted or “knocked out”, or the nucleotide sequence of the endogenous GXII PLA₂ gene has been mutated or the transcriptional regulatory region of the endogenous GXII PLA₂ gene has been altered).

A transgenic animal of the invention can be created by introducing GXII PLA₂-encoding nucleic acid into the male pronuclei of a fertilized oocyte, e.g., by microinjection, and allowing the oocyte to develop in a pseudopregnant female foster animal. A mouse GXII PLA₂ cDNA sequence (e.g., SEQ ID NO: 1) can be introduced as a transgene into the genome of a non-human animal (e.g., a mouse). Alternatively, a mammalian homologue of the mouse GXII PLA₂ gene, such as a human GXII PLA₂ gene, can be isolated based on hybridization to the mouse GXII PLA₂ cDNA and used as a transgene. Intronic sequences and polyadenylation signals can also be included in the transgene to increase the efficiency of expression of the transgene. A tissue-specific regulatory sequence(s) can be operably linked to the GXII PLA₂ transgene to direct expression of GXII PLA₂ protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the GXII PLA₂ transgene in its genome and/or expression of GXII PLA₂ mRNA in tissues or cells of the animals. A transgenic founder animal can then be used to breed additional animals carrying the transgene. Moreover, transgenic animals carrying a transgene encoding GXII PLA₂ can further be bred to other transgenic animals carrying other transgenes.

To create a homologous recombinant animal, a vector is prepared which contains at least a portion of a GXII PLA₂ gene into which a deletion, addition or substitution has been introduced to thereby alter, e.g., functionally disrupt, the endogenous GXII PLA₂ gene. The GXII PLA₂ gene preferably is a mouse GXII PLA₂ gene. For example, a mouse GXII PLA₂ gene can be isolated from a mouse genomic DNA library using a mouse GXII PLA₂ cDNA (e.g., SEQ ID NO: 1) as a probe. The mouse GXII PLA₂ gene then can be used to construct a homologous recombination vector suitable for altering an endogenous GXII PLA₂ gene in the mouse genome. In a preferred embodiment, the vector is designed such that, upon homologous recombination, the endogenous GXII PLA₂ gene is functionally disrupted (i.e., no longer encodes a functional protein; also referred to as a “knock out” vector). Alternatively, the vector can be designed such that, upon homologous recombination, the endogenous GXII PLA₂ gene is mutated or otherwise altered but still encodes functional protein (e.g., the upstream regulatory region can be altered to thereby alter the expression of the endogenous GXII PLA₂ protein). In the homologous recombination vector, the altered portion of the GXII PLA₂ gene is flanked at its 5′ and 3′ ends by additional nucleic acid of the GXII PLA₂ gene to allow for homologous recombination to occur between the exogenous GXII PLA₂ gene carried by the vector and an endogenous GXII PLA₂ gene in an embryonic stem cell. The additional flanking GXII PLA₂ nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced GXII PLA₂ gene has homologously recombined with the endogenous GXII PLA₂ gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a pseudopregnant foster mother and allowed to develop to term.

III. Isolated GXII PLA₂ Proteins and Anti-GXII PLA₂ Antibodies

Another aspect of the invention pertains to isolated GXII PLA₂ proteins, and portions thereof, such as biologically active portions, as well as peptide fragments suitable as immunogens to raise anti-GXII PLA₂ antibodies. In one embodiment, the invention provides an isolated preparation of GXII PLA₂ protein. Preferably, the GXII PLA₂ protein has an amino acid sequence shown in SEQ ID NO: 2, SEQ ID NO:4 or SEQ ID NO:6. In other embodiments, the GXII PLA₂ protein is substantially homologous to SEQ ID NO: 2, SEQ ID NO:4 or SEQ ID NO:6 and retains the functional activity of the protein of SEQ ID NO: 2, SEQ ID NO:4 or SEQ ID NO:6 yet differs in amino acid sequence due to natural allelic variation or mutagenesis, or is a homologue of the protein of SEQ ID NO: 2, SEQ ID NO:4 or SEQ ID NO:6 (e.g., a homologue from another mammalian species), as described in detail in subsection I above. Accordingly, in another embodiment, the GXII PLA₂ protein is a protein which comprises an amino acid sequence at least 60% homologous to the amino acid sequence of SEQ ID NO: 2, SEQ ID NO:4 or SEQ ID NO:6 and which selectively hydrolyzes arachidonic acid in the sn-2 position of phosphatidylethanolamine. Preferably, the protein is at least 70% homologous to SEQ ID NO: 2, SEQ ID NO:4 or SEQ ID NO:6, more preferably at least 80% homologous to SEQ ID NO: 2, SEQ ID NO:4 or SEQ ID NO:6, even more preferably at least 90% homologous to SEQ ID NO: 2 , SEQ ID NO:4 or SEQ ID NO:6, and most preferably at least 95%, 96%, 97%, 98%, 99% or 99.5% homologous to SEQ ID NO: 2, SEQ ID NO:4 or SEQ ID NO:6.

In other embodiments, the invention provides isolated portions of the GXII PLA₂ protein. For example, the invention further encompasses a portion of a GXII PLA₂ protein that retains the ability to hydrolyze arachidonic acid in the sn-2 position of phosphatidylethanolamine. As demonstrated in Example 3, GXII PLA₂ exhibits phospholipase A2 activity and shows a marked preference for arachidonic acid in the sn-2 position of phosphatidylethanolamine as a substrate. An in vitro hydrolysis assay (such as that described in Example 3) can be used to determine the ability of GXII PLA₂ peptide fragments to hydrolyze arachidonic acid in the sn-2 position of phosphatidylethanolamine to thereby identify peptide fragments of GXII PLA₂ that retain enzymatic activity.

GXII PLA₂ proteins are preferably produced by recombinant DNA techniques. For example, a nucleic acid molecule encoding the protein is cloned into an expression vector (as described above), the expression vector is introduced into a host cell (as described above) and the GXII PLA₂ protein is expressed in the host cell. The GXII PLA₂ protein can then be isolated from the cells by an appropriate purification scheme using standard protein purification techniques. Alternative to recombinant expression, a GXII PLA₂ polypeptide can be synthesized chemically using standard peptide synthesis techniques. Moreover, native GXII PLA₂ protein can be isolated from cells (e.g., from T cells), for example by immunoprecipitation using an anti-GXII PLA₂ antibody.

The invention also provides GXII PLA₂ fusion proteins. As used herein, a GXII PLA₂ “fusion protein” comprises a GXII PLA₂ polypeptide operatively linked to a non-GXII PLA₂ polypeptide. A “GXII PLA₂ polypeptide” refers to a polypeptide having an amino acid sequence corresponding to GXII PLA₂ protein, or a peptide fragment thereof, whereas a “non-GXII PLA₂ polypeptide” refers to a polypeptide having an amino acid sequence corresponding to another protein. Within the fusion protein, the term “operatively linked” is intended to indicate that the GXII PLA₂ polypeptide and the non-GXII PLA₂ polypeptide are fused in-frame to each other. The non-GXII PLA₂ polypeptide may be fused to the N-terminus or C-terminus of the GXII PLA₂ polypeptide. For example, in one embodiment, the fusion protein is a GST-GXII PLA₂ fusion protein in which the GXII PLA₂ sequences are fused to the C-terminus of the GST sequences. In another embodiment, the fusion protein comprises a GXII PLA₂ protein or peptide fused to green fluorescent protein (GFP). In yet another embodiment, the fusion protein is a GXII PLA₂-HA fusion protein in which the GXII PLA₂ nucleotide sequence is inserted in to the pCEP4-HA vector (Herrscher, R. F. et al. (1 995) Genes Dev. 9:3067-3082) such that the GXII PLA₂ sequences are fused in frame to an influenza hemagglutinin epitope tag. Such fusion proteins can facilitate the purification of recombinant GXII PLA₂, as well as detection of the fusion protein in host cells using an antibody that recognizes the epitope tag.

Preferably, a GXII PLA₂ fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence. Moreover, many expression vectors are commercially available that already encode a fusion moiety (e.g., a GST polypeptide or an HA epitope tag). A GXII PLA₂-encoding nucleic acid can be cloned into such an expression vector such that the fusion moiety is linked in-frame to the GXII PLA₂ protein.

An isolated GXII PLA₂ protein, or fragment thereof, can be used as an immunogen to generate antibodies that bind GXII PLA₂ using standard techniques for polyclonal and monoclonal antibody preparation. The GXII PLA₂ protein can be used to generate antibodies or, alternatively, an antigenic peptide fragment of GXII PLA₂ can be used as the immunogen. An antigenic peptide fragment of GXII PLA₂ typically comprises at least 8 amino acid residues, e.g., eight amino acid residues of the amino acid sequence shown in SEQ ID NO: 2, 4 or 6, and encompasses an epitope of GXII PLA₂ such that an antibody raised against the peptide forms a specific immune complex with GXII PLA₂. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of GXII PLA₂ that are located on the surface of the protein, e.g, hydrophilic regions. Hydrophobicity/hydrophilicity analysis of GXII PLA₂ protein sequences can be conducted to identify regions predicted to be located on the surface of the protein.

A GXII PLA₂ immunogen typically is used to prepare antibodies by immunizing a suitable subject, (e.g., rabbit, goat, mouse or other mammal) with the immunogen. An appropriate immunogenic preparation can contain, for examples, recombinantly expressed GXII PLA₂ protein or a chemically synthesized GXII PLA₂ peptide. The preparation can further include an adjuvant, such as Freund's complete or incomplete adjuvant, or similar immunostimulatory agent. Immunization of a suitable subject with an immunogenic GXII PLA₂ preparation induces a polyclonal anti-GXII PLA₂ antibody response.

Accordingly, another aspect of the invention pertains to anti-GXII PLA₂ antibodies. Polyclonal anti-GXII PLA₂ antibodies can be prepared as described above by immunizing a suitable subject with a GXII PLA₂ immunogen. The anti-GXII PLA₂ antibody titer in the immunized subject can be monitored over time by standard techniques, such as with an enzyme linked immunosorbent assay (ELISA) using immobilized GXII PLA₂. If desired, the antibody molecules directed against GXII PLA₂ can be isolated from the mammal (e.g., from the blood) and further purified by well known techniques, such as protein A chromatography to obtain the IgG fraction. At an appropriate time after immunization, e.g., when the anti-GXII PLA₂ antibody titers are highest, antibody-producing cells can be obtained from the subject and used to prepare monoclonal antibodies by standard techniques, such as the hybridoma technique originally described by Kohler and Milstein (1975, Nature 256:495-497) (see also, Brown et al. (1981) J. Immunol 127:539-46; Brown et al. (1980) J Biol Chem 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet., 3:231-36). Briefly, an immortal cell line (typically a myeloma) is fused to lymphocytes (typically splenocytes) from a mammal immunized with a GXII PLA₂ immunogen as described above, and the culture supernatants of the resulting hybridoma cells are screened to identify a hybridoma producing a monoclonal antibody that binds GXII PLA₂.

Any of the many well known protocols used for fusing lymphocytes and immortalized cell lines can be applied for the purpose of generating an anti-GXII PLA₂ monoclonal antibody (see, e.g., G. Galfre et al. (1977) Nature 266:55052; Gefter et al. Somatic Cell Genet., cited supra; Lemer, Yale J. Biol. Med., cited supra; Kenneth, Monoclonal Antibodies, cited supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, e.g, the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind GXII PLA₂, e.g, using a standard ELISA assay.

Alternative to preparing monoclonal antibody-secreting hybridomas, a monoclonal anti-GXII PLA₂ antibody can be identified and isolated by screening a recombinant combinatorial immunoglobulin library (e.g., an antibody phage display library) with GXII PLA₂ to thereby isolate immunoglobulin library members that bind GXII PLA₂. Kits for generating and screening phage display libraries are commercially available (e.g., the Pharmacia Recombinant Phage Antibody System, Catalog No. 27-9400-01; and the Stratagene SurfZAP.TM. Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; Barbas et al. (1991) PNAS 88:7978-7982; and McCafferty et al. Nature (1990) 348:552-554.

Additionally, recombinant anti-GXII PLA₂ antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira, et al. European Patent Application 184,187; Taniguchi, M., European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-GXII PLA₂ antibody (e.g., monoclonal antibody) can be used to isolate GXII PLA₂ by standard techniques, such as affinity chromatography or immunoprecipitation. An anti-GXII PLA₂ antibody can facilitate the purification of natural GXII PLA₂ from cells and of recombinantly produced GXII PLA₂ expressed in host cells. Moreover, an anti-GXII PLA₂ antibody can be used to detect GXII PLA₂ protein (e.g., in a cellular lysate or cell supernatant). Detection may be facilitated by coupling (i.e., physically linking) the antibody to a detectable substance. Accordingly, in one embodiment, an anti-GXII PLA₂ antibody of the invention is labeled with a detectable substance. Examples of detectable substances include various enzymes, prosthetic groups, fluorescent materials, luminescent materials and radioactive materials. Examples of suitable enzymes include horseradish peroxidase, alkaline phosphatase, .beta.-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include .sup.125 I, .sup.131 I, .sup.35 S or .sup.3 H.

IV. Pharmaceutical Compositions

The GXII PLA₂ proteins and anti-GXII PLA₂ antibodies of the invention can be incorporated into pharmaceutical compositions suitable for administration. Such compositions typically comprise the protein or antibody and a pharmaceutically acceptable carrier. As used herein the term “pharmaceutically acceptable carrier” is intended to include any and all solvents, dispersion media, coatings, antibacterial and antifingal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL.TM. (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a GXII PLA₂ protein or anti-GXII PLA₂ antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811.

V. Methods of the Invention

Another aspect of the invention pertains to a method of using the various GXII PLA₂ compositions of the invention. For example, the invention provides a method for detecting the presence of GXII PLA₂ protein or MRNA in a biological sample. The method involves contacting the biological sample with an agent capable of detecting GXII PLA₂ protein or mRNA such that the presence of GXII PLA₂ protein or MRNA is detected in the biological sample. A preferred agent for detecting GXII PLA₂ mRNA is a labeled nucleic acid probe capable of hybridizing to GXII PLA₂ mRNA. The nucleic acid probe can be, for example, a GXII PLA₂ cDNA, e.g., SEQ ID NO: 1, SEQ ID NO:3 or SEQ ID NO:5, or a portion thereof, such as an oligonucleotide of at least 15, 30, 50, 100, 200, 300, 400, 500, 600, 700, 800, 900 or 1000 nucleotides in length and sufficient to specifically hybridize under stringent conditions to GXII PLA₂ mRNA. A preferred agent for detecting GXII PLA₂ protein is a labeled antibody capable of binding to GXII PLA₂ protein. Antibodies can be polyclonal, or more preferably, monoclonal. An intact antibody, or a fragment thereof (e.g., Fab or F(ab′).sub.2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids. For example, techniques for detection of GXII PLA₂ mRNA include Northern hybridizations and in situ hybridizations. Techniques for detection of GXII PLA₂ protein include enzyme linked immunosorbent assays (ELISAs), Western blots, immunoprecipitations and immunofluorescence.

Another aspect of the invention pertains to screening assays that allow for the identification of compounds capable of modulating the activity of a GXII PLA₂ protein of the invention. In one embodiment, the invention provides a method for identifying a compound that modulates the activity of GXII PLA₂, comprising providing an indicator composition comprising GXII PLA₂ and a substrate for GXII PLA₂;

contacting the indicator composition with a test compound; and

determining the effect of the test compound on GXII PLA₂ activity toward the substrate in the indicator composition to thereby identify a compound that modulates the activity of GXII PLA₂.

Preferably, the substrate is arachidonic acid in the sn-2 position of phosphatidylethanolamine, for which the GXII PLA₂ proteins of the invention exhibit a preference as a substrate. However, other substrates towards which GXII PLA₂ shows at least detectable activity could also be used in the screening assay, although the sensitivity of the assay would be diminished using a non-optimal substrate. The reaction conditions under which the test compound is assessed preferably are a basic pH in millimolar calcium. The GXII PLA₂ used in the indicator composition of the assay can be, for example, recombinantly produced GXII PLA₂ protein, purified protein isolated from appropriate cells (e.g., Th2 cells), cell lysates that contain GXII PLA₂ (e.g., cell lysate from Th2 cells that express GXII PLA₂) or cellular compositions that contain GXII PLA₂ (e.g., Th2 cell compositions). To determine the effect of the test compound on GXII PLA₂ activity toward the substrate, the amount of GXII PLA₂ activity in the presence of the test compound can be compared to the amount of GXII PLA₂ activity in the absence of the test compound. Alternatively, to determine the effect of the test compound on GXII PLA₂ activity toward the substrate, the amount of GXII PLA₂ activity in the presence of the test compound can be compared to the amount of GXII PLA₂ activity in the presence of a difference, control compound. Alternatively, to determine the effect of the test compound on GXII PLA₂ activity toward the substrate, the amount of GXII PLA₂ activity in the presence of the test compound can be compared to the amount of activity of another PLA₂ (e.g., GV PLA₂) in the presence of the same test compound and same substrate (to thereby provide an assessment of the enzymatic specificity of the test compound). Test compounds that decrease GXII PLA₂ activity as compared to an appropriate control are thus identified as inhibitory modulators of GXII PLA₂, whereas test compounds that increase GXII PLA₂ activity as compared to an appropriate control are thus identified as stimulatory modulators of GXII PLA₂. An nonlimiting example of an assay suitable for use in the screening assays of the invention to test compounds is described in further detail in Example 3.

Yet another aspect of the invention pertains to methods of modulating GXII PLA₂ activity in a cell. The modulatory methods of the invention involve contacting the cell with an agent that modulates GXII PLA₂ activity such that GXII PLA₂ activity in the cell is modulated. The agent may act by modulating the activity of GXII PLA₂ protein in the cell or by modulating transcription of the GXII PLA₂ gene or translation of the GXII PLA₂ mRNA. As used herein, the term “modulating” is intended to include inhibiting or decreasing GXII PLA₂ activity and stimulating or increasing GXII PLA₂ activity. Accordingly, in one embodiment, the agent inhibits GXII PLA₂ activity. An inhibitory agent may function, for example, by directly inhibiting GXII PLA₂ activity or by inhibiting an interaction between GXII PLA₂ and its substrate. In another embodiment, the agent stimulates GXII PLA₂ activity. A stimulatory agent may function, for example, by directly stimulating GXII PLA₂ activity or by promoting an interaction between GXII PLA₂ and its substrate.

A preferred stimulatory agent for stimulating GXII PLA₂ in a cell is a nucleic acid molecule encoding GXII PLA₂, wherein the nucleic acid molecule is introduced into the cell in a form suitable for expression of GXII PLA₂ in the cell. For example, a GXII PLA₂ cDNA is cloned into a recombinant expression vector and the vector is transfected into the cell. To express a GXII PLA₂ in a cell, typically a GXII PLA₂ cDNA is first introduced into a recombinant expression vector using standard molecular biology techniques (see e.g., sections I and II above). Another form of a stimulatory agent for stimulating GXII PLA₂ activity in a cell is a chemical compound that stimulates the expression or activity of an GXII PLA₂ protein in the cell. Such compounds can be identified using screening assays that select for compounds that stimulate the expression or activity of GXII PLA₂, such as screening assays described above.

Inhibitory agents of the invention for inhibiting GXII PLA₂ activity can be, for example, intracellular binding molecules that act to inhibit the expression or activity of GXII PLA₂. As used herein, the term “intracellular binding molecule” is intended to include molecules that act intracellularly to inhibit the expression or activity of a protein by binding to the protein or to a nucleic acid (e.g., an mRNA molecule) that encodes the protein. Examples of intracellular binding molecules include antisense nucleic acids and antibodies (e.g., intracellular antibodies). In one embodiment, an inhibitory agent of the invention is an antisense nucleic acid molecule that is complementary to a gene encoding GXII PLA₂, or to a portion of said gene, or a recombinant expression vector encoding said antisense nucleic acid molecule. The use of antisense nucleic acids to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an MRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA. An antisense nucleic acid for inhibiting the expression of a GXII PLA₂ protein in a cell can be designed based upon the nucleotide sequence encoding the GXII PLA₂, constructed according to the rules of Watson and Crick base pairing, as described in further detail in section I above.

An antisense nucleic acid can exist in a variety of different forms. For example, the antisense nucleic acid can be an oligonucleotide that is complementary to only a portion of a GXII PLA₂ gene. An antisense oligonucleotides can be constructed using chemical synthesis procedures known in the art. An antisense oligonucleotide can be chemically synthesized using naturally occurring nucleotides or variously modified nucleotides designed to increase the biological stability of the molecules or to increase the physical stability of the duplex formed between the antisense and sense nucleic acids, e.g. phosphorothioate derivatives and acridine substituted nucleotides can be used. To inhibit GXII PLA₂ protein expression in cells in culture, one or more antisense oligonucleotides can be added to cells in culture media, typically at 200 μg oligonucleotide/ml.

Alternatively, an antisense nucleic acid can be produced biologically using an expression vector into which a nucleic acid has been subcloned in an antisense orientation (i e, nucleic acid transcribed from the inserted nucleic acid will be of an antisense orientation to a target nucleic acid of interest). Regulatory sequences operatively linked to a nucleic acid cloned in the antisense orientation can be chosen which direct the expression of the antisense RNA molecule in a cell of interest, for instance promoters and/or enhancers or other regulatory sequences can be chosen which direct constitutive, tissue specific or inducible expression of antisense RNA. The antisense expression vector is prepared as described above for recombinant expression vectors, except that the cDNA (or portion thereof) is cloned into the vector in the antisense orientation. The antisense expression vector can be in the form of, for example, a recombinant plasmid, phagemid or attenuated virus. The antisense expression vector is introduced into cells using a standard transfection technique, as described above for recombinant expression vectors. In another embodiment, an antisense nucleic acid for use as an inhibitory agent is a ribozyme, as described above in section I.

Another type of inhibitory agent that can be used to inhibit the expression and/or activity of a GXII PLA₂ protein in a cell is an intracellular antibody specific for the GXII PLA₂protein. The use of intracellular antibodies to inhibit protein function in a cell is known in the art (see e.g., Carlson, J. R. (1988) Mol. Cell. Biol. 8:2638-2646; Biocca, S. et al (1990) EMBO J. 9:101-108; Werge, T. M. et al. (1990) FEBS Letters 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Bio/Technology 12:396-399; Chen, S-Y. et al. (1994) Human Gene Therapy 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al.).

To inhibit protein activity using an intracellular antibody, a recombinant expression vector is prepared which encodes the antibody chains in a form such that, upon introduction of the vector into a cell, the antibody chains are expressed as a functional antibody in an intracellular compartment of the cell. For inhibition of GXII PLA₂ activity according to the inhibitory methods of the invention, preferably an intracellular antibody that specifically binds the GXII PLA₂ is expressed within the cell at a location where GXII PLA₂ found in the cell. The subcellular localization of GXII PLA₂ has been studied and is described in further detail in Example 4. To prepare an intracellular antibody expression vector, antibody light and heavy chain cDNAs encoding antibody chains specific for the target protein of interest, e.g., a GXII PLA₂ protein, are isolated, typically from a hybridoma that secretes a monoclonal antibody specific for the GXII PLA₂ protein or by screening a recombinant combinatorial immunoglobulin library (e.g., an antibody phage display library) with a GXII PLA₂ protein or peptide to thereby isolate immunoglobulin library members that bind specifically to a GXII PLA₂ protein. Once a monoclonal antibody specific for the GXII PLA₂ protein has been identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or CDNA library screening. For recombinant antibodies, such as from a phage display library, cDNA encoding the light and heavy chains can be recovered from the display package (e.g., phage) isolated during the library screening process. Nucleotide sequences of antibody light and heavy chain genes from which PCR primers or cDNA library probes can be prepared are known in the art. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database.

Once obtained, the antibody light and heavy chain sequences are cloned into a recombinant expression vector using standard methods. The expression vector can encode an intracellular antibody in one of several different forms. For example, in one embodiment, the vector encodes full-length antibody light and heavy chains such that a full-length antibody is expressed intracellularly. In another embodiment, the vector encodes a fIll-length light chain but only the VH/CH1 region of the heavy chain such that a Fab fragment is expressed intracellularly. In the most preferred embodiment, the vector encodes a single chain antibody (scFv) wherein the variable regions of the light and heavy chains are linked by a flexible peptide linker (e.g., (Gly₄Ser)₃) and expressed as a single chain molecule. To inhibit GXII PLA₂ activity in a cell, the expression vector encoding the GXII PLA₂-specific intracellular antibody is introduced into the cell by standard transfection methods, as discussed hereinbefore.

Yet another type of inhibitory agent that can be used to inhibit the expression and/or activity of a GXII PLA₂ protein in a cell is chemical compound that inhibits the expression or activity of an endogenous GXII PLA₂ protein in the cell. Such compounds can be identified using screening assays that select for compounds that inhibit the expression or activity of aGXII PLA₂, such as screening assays described in further detail above.

The method of the invention for modulating GXII PLA₂ activity can be practiced either in vitro or in vivo. For practicing the method in vitro, cells can be obtained from a subject by standard methods and incubated (i.e., cultured) in vitro with a stimulatory or inhibitory agent of the invention to stimulate or inhibit, respectively, GXII PLA₂ activity. For example, peripheral blood mononuclear cells (PBMCs) can be obtained from a subject and isolated by density gradient centrifugation, e.g., with Ficoll/Hypaque. Specific cell populations can be depleted or enriched using standard methods. For example, monocytes/macrophages can be isolated by adherence on plastic. T cells or B cells can be enriched or depleted, for example, by positive and/or negative selection using antibodies to T cell or B cell surface markers, for example by incubating cells with a specific primary monoclonal antibody (mAb), followed by isolation of cells that bind the mAb using magnetic beads coated with a secondary antibody that binds the primary mAb. Peripheral blood or bone marrow derived hematopoietic stem cells can be isolated by similar techniques using stem cell-specific mabs (e.g., anti-CD34 mabs). Specific cell populations (e.g., Th2 cells) can also be isolated by fluoresence activated cell sorting according to standard methods. Monoclonal antibodies to cell-specific surface markers known in the art and many are commercially available.

Moreover, cells (e.g., Th2 cells) treated in vitro with either a stimulatory or inhibitory agent can be administered to a subject, e.g., to modulate Th2 responses in the subject. Accordingly, in another embodiment, the method of the invention for modulating GXII PLA2 activity by Th2 cells further comprises administering the Th2 cells to a subject to thereby modulate Th2 responses in a subject. For administration to a subject, it may be preferable to first remove residual agents in the culture from the cells before administering them to the subject. This can be done for example by a Ficoll/Hypaque gradient centrifugation of the cells. For further discussion of ex vivo genetic modification of cells followed by readministration to a subject, see also U.S. Pat. No. 5,399,346 by W. F. Anderson et al.

Alternatively, the methods of the invention for modulating GXII PLA2 activity can be carried out in vivo in a subject. The term “subject” is intended to include living organisms in which an immune response can be elicited. Preferred subjects are mammals. Examples of subjects include humans, monkeys, dogs, cats, mice, rats, cows, horses, goats and sheep. GXII PLA₂ activity can be modulated in a subject by administering to the subject a modulatory agent, e.g., stimulatory or inhibitory agent, as described above. For stimulatory or inhibitory agents that comprise nucleic acids (including recombinant expression vectors encoding GXII PLA₂, antisense RNA or intracellular antibodies), the agents can be introduced into cells of the subject using methods known in the art for introducing nucleic acid (e.g., DNA) into cells in vivo. Examples of such methods include:

Direct Injection: Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo can be used. Such an apparatus is commercially available (e.g., from BioRad).

Receptor-Mediated DNA Uptake: Naked DNA can also be introduced nto cells in vivo by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and .S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Retroviruses: Defective retroviruses are well characterized for use in gene transfer for gene therapy purposes (for a review see Miller, A. D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include .psi.Crip, .psi.Cre, .psi.2 and .psi.Am. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral El and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay.

A modulatory agent, such as a chemical compound that stimulates or inhibits GXII PLA₂ activity, can be administered to a subject as a pharmaceutical composition. Such compositions typically comprise the modulatory agent and a pharmaceutically acceptable carrier, as described in further detail above. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The modulatory methods of the invention can be used for a variety of purposes. For example, agents that modulate GXII PLA₂ activity can be used to modulate Th2 cell differentiation and/or activity, since GXII PLA₂ has been shown to be preferentially expressed in Th2 cells and there is considerable evidence for the integral role of arachidonic acid metabolites in the differentiation of Th2 cells. For example, PGE2 is an inhibitor of Th1 cytokines such as IL-2, IL-12 and IFN-γ while simultaneously encouraging the production of the Th2 cytokines IL-4, IL-5 and IL-13. Furthermore, PGE2 directly inhibits the transcription of IL-2 genes but not IL-4 genes in human Jurkat T cells. Thus, a variety of evidence indicates that CD4+ helper T cells are likely to differentiate into Th2 cells in the presence of prostaglandin. Agents that stimulate GXII PLA₂ activity (i.e., stimulatory agents of the invention) can be used to increase prostaglandin production to thereby favor development of a Th2 cell response, whereas agents that inhibit GXII PLA₂ activity (i.e., inhibitory agents of the invention) can be used to inhibit prostaglandin production to thereby favor development of a Th1 cell response

Furthermore, as described further in the Examples section below, GV PLA₂ also has now been shown to be preferentially expressed in Th2 cells and thus agents that modulate GV PLA₂ activity also can be used to modulate prostaglandin production by T cells and thereby modulate Th2 cell differentiation and/or activity. (Stimulatory and inhibitory agents specific for GV PLA₂ can be prepared as described above for stimulatory and inhibitory agents specific for GXII PLA₂, using the nucleotide and/or amino acid sequence of GV PLA₂ that is available in the art). Thus, in another aspect, the invention provides a method for modulating Th2 cell differentiation and/or activity comprising contacting Th2 cells, or precursors thereof, with a modulator of GXII PLA₂ or GV PLA₂ such thatTh2 cell differentiation or activity is modulated.

Numerous disease conditions associated with a predominant Th1 or Th2-type response have been identified and could benefit from modulation of the type of T helper cell response mounted in the individual suffering from the disease condition. Examples of such diseases include:

A. Allergies

Allergies are mediated through IgE antibodies whose production is regulated by the activity of Th2 cells and the cytokines produced thereby. In allergic reactions, IL-4 is produced by Th2 cells, which further stimulates production of IgE antibodies and activation of cells that mediate allergic reactions, i e., mast cells and basophils. IL-4 also plays an important role in eosinophil mediated inflammatory reactions. Accordingly, inhibitors of GXII PLA₂ or GV PLA₂ can be used to inhibit prostaglandin production to thereby inhibit the production of Th2-associated cytokines, and in particular IL-4, in allergic patients as a means to downregulate production of pathogenic IgE antibodies. An inhibitory agent of the invention may be directly administered to the subject or cells (e.g., Thp cells or Th2 cells) may be obtained from the subject, contacted with an inhibitory agent ex vivo, and readministered to the subject. Moreover, in certain situations it may be beneficial to coadminister to the subject the allergen together with the inhibitory agent or cells treated with the inhibitory agent to inhibit (e.g., desensitize) the allergen-specific response. The treatment may be further enhanced by administering other Th1-promoting agents, such as the cytokine IL-12 or antibodies to Th2-associated cytokines (e.g., anti-IL-4 antibodies), to the allergic subject in amounts sufficient to further stimulate a Th1-type response.

B. Cancer

The expression of Th2-promoting cytokines has been reported to be elevated in cancer patients (see e.g., Yamamura, M., et al. (1993) J. Clin. Invest. 91:1005-1010; Pisa, P., et al. (1992) Proc. Natl. Acad. Sci USA 89:7708-7712) and malignant disease is often associated with a shift from Th1 type responses to Th2 type responses along with a worsening of the course of the disease. Accordingly, inhibitors of GXII PLA₂ or GV PLA₂ can be used to inhibit prostaglandin production to thereby inhibit the production of Th2-associated cytokines in cancer patients, as a means to counteract the Th1 to Th2 shift and thereby promote an ongoing Th1 response in the patients to ameliorate the course of the disease. The inhibitory method can involve either direct administration of an inhibitory agent to a subject with cancer or ex vivo treatment of cells obtained from the subject (e.g., Thp or Th2 cells) with an inhibitory agent followed by readministration of the cells to the subject. The treatment may be further enhanced by administering other Th1-promoting agents, such as the cytokine IL-12 or antibodies to Th2-associated cytokines (e.g., anti-IL-4 antibodies), to the recipient in amounts sufficient to further stimulate a Th1-type response.

C. Infectious Diseases

The expression of Th2-promoting cytokines also has been reported to increase during a variety of infectious diseases, including HIV infection, tuberculosis, leishmaniasis, schistosomiasis, filarial nematode infection and intestinal nematode infection (see e.g.; Shearer, G. M. and Clerici, M. (1992) Prog. Chem. Immunol. 54:21-43; Clerici, M and Shearer, G. M. (1993) Immunology Today 14:107-111; Fauci, A. S. (1988) Science 239:617623; Locksley, R. M. and Scott, P. (1992) Immunoparasitology Today 1:A58-A61; Pearce, E. J., et al. (1991) J. Exp. Med. 173:159-166; Grzych, J-M., et al. (1991) J. Immunol. 141:1322-1327; Kullberg, M. C., et al. (1992) J. Immunol. 148:3264-3270; Bancroft, A. J., et al. (1993) J. Immunol. 150:1395-1402; Pearlman, E., et al. (1993) Infect. Immun. 61:1105-1112; Else, K. J., et al (1994) J. Exp. Med. 179:347-351) and such infectious diseases are also associated with a Th1 to Th2 shift in the immune response. Accordingly, inhibitors of GXII PLA₂ or GV PLA₂ can be used to inhibit prostaglandin production to thereby inhibit the production of Th2-associated cytokines in subjects with infectious diseases, as a means to counteract the Th1 to Th2 shift and thereby promote an ongoing Th1 response in the patients to ameliorate the course of the infection. The inhibitory method can involve either direct administration of an inhibitory agent to a subject with an infectious disease or ex vivo treatment of cells obtained from the subject (e.g., Thp or Th2 cells) with an inhibitory agent followed by readministration of the cells to the subject. The treatment may be further enhanced by administering other Th1-promoting agents, such as the cytokine IL-12 or antibodies to Th2-associated cytokines (e.g., anti-IL-4 antibodies), to the recipient in amounts sufficient to further stimulate a Th1-type response.

D. Autoimmune Diseases

The stimulatory methods of the invention can be used therapeutically in the treatment of autoimmune diseases that are associated with a Th2-type dysfunction. Many autoimmune disorders are the result of inappropriate activation of T cells that are reactive against self tissue and that promote the production of cytokines and autoantibodies involved in the pathology of the diseases. Modulation of T helper-type responses can have an effect on the course of the autoimmune disease. For example, in experimental allergic encephalomyelitis (EAE), stimulation of a Th2-type response by administration of IL-4 at the time of the induction of the disease diminishes the intensity of the autoimmune disease (Paul, W. E., et al. (1994) Cell 76:241-251). Furthermore, recovery of the animals from the disease has been shown to be associated with an increase in a Th2-type response as evidenced by an increase of Th2-specific cytokines (Koury, S. J., et al. (1992) J. Exp. Med. 176:1355-1364). Moreover, T cells that can suppress EAE secrete Th2-specific cytokines (Chen, C., et al. (1994) Immunity 1:147-154). Since stimulation of a Th2-type response in EAE has a protective effect against the disease, stimulation of a Th2 response in subjects with multiple sclerosis (for which EAE is a model) is likely to be beneficial therapeutically.

Similarly, stimulation of a Th2-type response in type I diabetes in mice provides a protective effect against the disease. Indeed, treatment of NOD mice with IL-4 (which promotes a Th2 response) prevents or delays onset of type I diabetes that normally develops in these mice (Rapoport, M. J., et al. (1993) J. Exp. Med. 178:87-99). Thus, stimulation of a Th2 response in a subject suffering from or susceptible to diabetes may ameliorate the effects of the disease or inhibit the onset of the disease.

Yet another autoimmune disease in which stimulation of a Th2-type response may be beneficial is rheumatoid arthritis (RA). Studies have shown that patients with rheumatoid arthritis have predominantly Thl cells in synovial tissue (Simon, A. K., et al., (1994) Proc. Natl. Acad. Sci USA 91:8562-8566). By stimulating a Th2 response in a subject with RA, the detrimental Th1 response can be concomitantly downmodulated to thereby ameliorate the effects of the disease. Accordingly, stimulators of GXII PLA₂ or GV PLA₂ can be used to stimulate prostaglandin production to thereby stimulate production of Th2-associated cytokines in subjects suffering from, or susceptible to, an autoimmune disease in which a Th2-type response is beneficial to the course of the disease. The stimulatory method can involve either direct administration of a stimulatory agent to the subject or ex vivo treatment of cells obtained from the subject (e.g., Thp, Th1 cells, B cells, non-lymphoid cells) with a stimulatory agent followed by readministration of the cells to the subject. The treatment may be further enhanced by administering other Th2-promoting agents, such as IL-4 itself or antibodies to Th1-associated cytokines, to the subject in amounts sufficient to further stimulate a Th2-type response.

In contrast to the autoimmune diseases described above in which a Th2 response is desirable, other autoimmune diseases may be ameliorated by a Th1-type response. Such diseases can be treated using an inhibitory agent of the invention (as described above for cancer and infectious diseases). The treatment may be further enhanced by administrating a Th1-promoting cytokine (e.g., IFN-.gamma.) to the subject in amounts sufficient to further stimulate a Th1-type response.

The efficacy of agents for treating autoimmune diseases can be tested in the above described animal models of human diseases (e.g., EAE as a model of multiple sclerosis and the NOD mice as a model for diabetes) or other well characterized animal models of human autoimmune diseases. Such animal models include the mrl/lpr/lpr mouse as a model for lupus erythematosus, murine collagen-induced arthritis as a model for rheumatoid arthritis, and murine experimental myasthenia gravis (see Paul ed., Fundamental Immunology, Raven Press, New York, 1989, pp. 840-856). A modulatory (ie., stimulatory or inhibitory) agent of the invention is administered to test animals and the course of the disease in the test animals is then monitored by the standard methods for the particular model being used. Effectiveness of the modulatory agent is evidenced by amelioration of the disease condition in animals treated with the agent as compared to untreated animals (or animals treated with a control agent).

Non-limiting examples of autoimmune diseases and disorders having an autoimmune component that may be treated according to the invention include diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

E. Transplantation

While graft rejection or graft acceptance may not be attributable exclusively to the action of a particular T cell subset (i.e., Th1 or Th2 cells) in the graft recipient (for a discussion see Dallman, M. J. (1995) Curr. Opin. Immunol. 7:632-638), numerous studies have implicated a predominant Th2 response in prolonged graft survival or a predominant Th1 response in graft rejection. For example, graft acceptance has been associated with production of a Th2 cytokine pattern and/or graft rejection has been associated with production of a Th1 cytokine pattern (see e.g., Takeuchi, T. et al. (1992) Transplantation 53:1281-1291; Tzakis, A. G. et al. (1994) J. Pediatr. Surg 29:754-756; Thai, N. L. et al. (1995) Transplantation 59:274-281). Additionally, adoptive transfer of cells having a Th2 cytokine phenotype prolongs skin graft survival (Maeda, H. et al. (1994) Int. Immunol. 6:855-862) and reduces graft-versus-host disease (Fowler, D. H. et al. (1994) Blood 84:3540-3549; Fowler, D. H. et al. (1994) Prog. Clin. Biol. Res. 389:533-540). Still further, administration of IL-4, which promotes Th2 differentiation, prolongs cardiac allograft survival (Levy, A. E. and Alexander, J. W. (1995) Transplantation 60:405-406), whereas administration of IL-12 in combination with anti-IL-10 antibodies, which promotes Th1 differentiation, enhances skin allograft rejection (Gorczynski, R. M. et al. (1995) Transplantation 60:1337-1341).

Accordingly, stimulators of GXII PLA₂ or GV PLA₂ can be used to stimulate prostaglandin production to thereby stimulate production of Th2-associated cytokines in transplant recipients to prolong survival of the graft. The stimulatory methods can be used both in solid organ transplantation and in bone marrow transplantation (e.g., to inhibit graft-versus-host disease). The stimulatory method can involve either direct administration of a stimulatory agent to the transplant recipient or ex vivo treatment of cells obtained from the subject (e.g., Thp, Th1 cells, B cells, non-lymphoid cells) with a stimulatory agent followed by readministration of the cells to the subject. The treatment may be further enhanced by administering other Th2-promoting agents, such as IL-4 itself or antibodies to Th1-associated cytokines, to the recipient in amounts sufficient to further stimulate a Th2-type response.

In addition to the foregoing disease situations, the modulatory methods of the invention also are useful for other purposes. For example, the stimulatory methods of the invention (i.e., methods using a stimulatory agent) can be used to stimulate production of Th2-promoting cytokines (e.g., IL-4)in vitro for commercial production of these cytokines (e.g., cells can be contacted with the stimulatory agent in vitro to stimulate IL-4 production and the IL-4 can be recovered from the culture supernatant, further purified if necessary, and packaged for commercial use).

Furthermore, the modulatory methods of the invention can be applied to vaccinations to promote either a Th1 or a Th2 response to an antigen of interest in a subject. That is, the agents of the invention can serve as adjuvants to direct an immune response to a vaccine either to a Th1 response or a Th2 response. For example, to stimulate an antibody response to an antigen of interest (i e., for vaccination purposes), the antigen and a stimulatory agent of the invention can be coadministered to a subject to promote a Th2 response to the antigen in the subject, since Th2 responses provide efficient B cell help and promote IgG1 production. Alternatively, to promote a cellular immune response to an antigen of interest, the antigen and an inhibitory agent of the invention can be coadministered to a subject to promote a Th1 response to an antigen in a subject, since Th1 responses favor the development of cell-mediated immune responses (e.g., delayed hypersensitivity responses). The antigen of interest and the modulatory agent can be formulated together into a single pharmaceutical composition or in separate compositions. In a preferred embodiment, the antigen of interest and the modulatory agent are administered simultaneously to the subject. Alternatively, in certain situations it may be desirable to administer the antigen first and then the modulatory agent or vice versa (for example, in the case of an antigen that naturally evokes a Th1 response, it may be beneficial to first administer the antigen alone to stimulate a Th1 response and then administer a stimulatory agent, alone or together with a boost of antigen, to shift the immune response to a Th2 response).

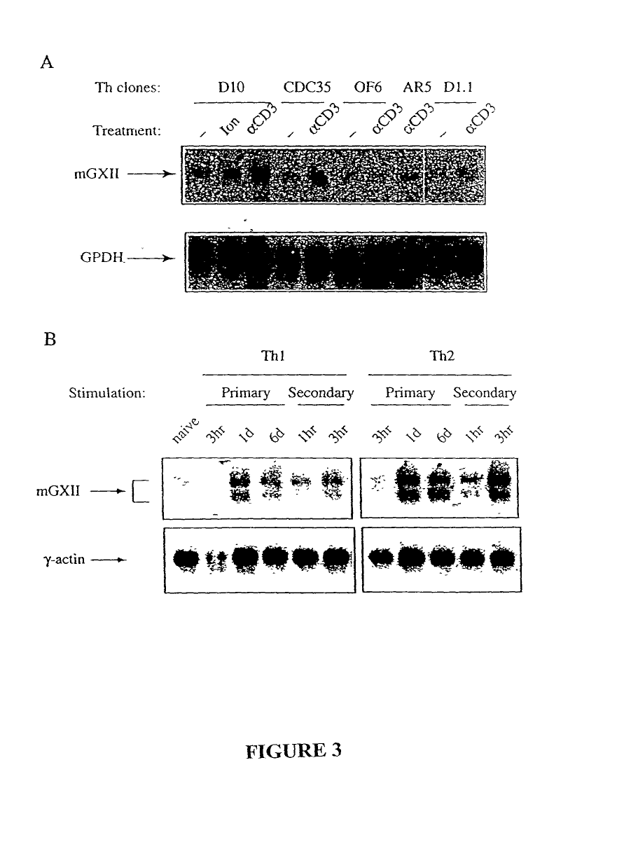

Furthermore, in light of the report that COX2 and hPGDS are also preferentially expressed in Th2 cells (Tanaka, K. et al. (2000) J. Immunol. 164:2277-2280), it is reasonable to predict that the Th2 cell-specific PLA₂s, such as GXII and GV, are functionally linked to COX2 and HPGDS in Th2 cells. Thus, in Th2 cells, GXII PLA₂, and GV PLA₂ may provide arachidonic acid to COX2 for generation of intermediates which hPGDS converts to PGD2. This scenario is further supported by three observations. First, GXII PLA₂ was induced in Th2 cells after TCR stimulation (FIG. 3B) in a time course parallel to that observed for COX2 induction (Tanaka, K. et al. cited supra). Second, exogenous GV PLA₂ could induce the expression of COX2 in macrophages (Balsinde, J. et al. (1999) J. Biol. Chem. 274:25967-25970). Third, mGXII-1 PLA₂ is present in a perinuclear location in transfected BHK cells. It is known that the generation of eicosanoids is dependent on the location within the cell of the enzymes of their biosynthesis, which appears to occur in a perinuclear location. Group V PLA₂ has been described in association with the nuclear envelope in mouse mast cells and acts in a co-operative manner with GIV cPLA₂ to provide arachidonic acid for eicosanoid biosynthesis (Bingham, C. O. et al. (1999) J. Biol. Chem. 274:31476-31484). Thus, GXII PLA₂ and GV PLA₂, two Th2 cell-specific enzymes, are well placed for a functional interaction with COX2 induced at the nuclear envelope and endoplasmic reticulum to generate PGG2/PGH2 which are processed by hPGDS to provide PGD2 and its metabolites. Interestingly, a metabolite of PGD2, 15-deoxy-Δ^{12,14}-PGJ₂ (15d-PGJ₂) is one of the natural ligands of the nuclear co-receptor, peroxisome proliferator-activated receptor-γ (PPARγ) (Forman, B. M. et al. (1995) Cell 83:803-812; Kliewer, S. A. et al. (1995) Cell 83:813-819), which was recently shown to be induced by the Th2 cytokine, IL-4, and which can regulate gene expression in both T and non-T cells (Huang, J. T. et al. (1999) Nature 400:378-382; Clark, R. B. et al. (2000) J. Immunol. 164:1364-1371). Also, GXII PLA₂ and GV PLA₂ might affect the differentiation and function of Th2 cells independent of their enzymatic activities. In agreement with this hypothesis, GI and GIIA PLA₂s can initiate signaling events by binding to lectin-like cell surface receptors (Cupillard, L. et al. (1997) J. Biol. Chem. 272:15745-15752; Copic, A. et al. (1999) J. Biol. Chem. 274:26315-26320).

Accordingly, in another aspect, the invention provides a method for modulating prostaglandin production by Th2 cells comprising contacting Th2 cells with a modulator of GXII PLA₂ or GV PLA₂ activity such that prostaglandin production by the Th2 cells is modulated. The modulator may be a stimulatory agent (i.e., an agent that stimulates GXII PLA₂ or GV PLA₂ activity) or an inhibitory agent (i.e., an agent that inhibits GXII PLA₂ or GV PLA₂ activity), as described above. Prostaglandin production may be modulated in vitro, by contacting the modulator with Th2 cells in vitro, or in vivo by administering the modulator to a subject, as described above.

Numerous disease conditions and disorders associated with prostaglandin production have been identified and could benefit from modulation of GXII PLA₂ or GV PLA₂ activity in the individual suffering from the disease condition or disorder. Examples of such diseases and conditions include pain, inflammation, arthritis (such as rheumatoid arthritis, reactive arthritis, osteoarthritis), autoimmune diseases (such as type 1 diabetes, multiple sclerosis) and neurological disorders (e.g., stroke, Alzheimer's disease). Furthermore, prostaglandins have been found to play a role in uterine implantation of the embryo. Thus, modulators of GXII PLA₂ or GV PLA₂ activity may be beneficial in the treatment of infertility, as well as for contraceptive uses.

This invention is further illustrated by the following examples which should not be construed as limiting. The contents of all references, patents and published patent applications cited throughout this application are hereby incorporated by reference. Nucleotide and amino acid sequences deposited in public databases as referred to herein are also hereby in corporated by reference.

The following Materials and Methods were used in the examples:

Cell Culture

Murine Th2 clones, D10 and CDC35, and murine Th1 clones, AE7, OF.6 and AR5 were maintained in RPMI supplemented with 10% FCS and 10% conditioned medium from Con A stimulated rat splenocytes. The murine Th1 clone D1.1 was maintained in RPMI supplemented with 10% FCS, 5% T-STIM (Collaborative Biomedical, Bedford, Mass.), and human IL-2 at 50 unit/ml. When indicated, Th clones were stimulated with plate bound anti-CD3 for 6 hours before harvesting. The BHK cell is a baby hamster kidney cell line maintained in DMEM supplemented with 10% FCS.

In vitro Differentiation and Stimulation of CD4+Th and B Cells

CD4+Th cells and B cells were purified from spleens and lymph nodes harvested from 6-8 week-old Balb/C mice by using CD4 (L3T4) and CD45R (B220) MicroBeads (Miltenyi Biotec Inc. Auburn, Calif.) according to the manufacturer's instruction. Purified CD4+Th cells were stimulated in vitro with plate-bound anti-CD3 mAb (2C11) at 1 μg/ml in the presence of anti-IL-12 mAb (5C3) at 20 μg/ml (Th2 skewing conditions) or anti-IL-4 mAb (11B11) at 5 μg /ml (Th1 skewing conditions). Twenty-four hours poststimulation, IL-2 at 50 units/ml was added to all cultures. In addition, IL-4 at 500 units/ml or IL-12 at 50 units/ml was added into Th2 or Thl cultures respectively. Seven days post stimulation, cells were harvested, washed thoroughly, and restimulated with plate-bound anti-CD3. Twenty-four hours post restimulation, cells were harvested for RNA preparation. All antibodies were purchased from PharMingen (San Diego, CA). The purified B cells were stimulated with PMA (50 ng/ml) and Ionomycin (1 μM) for 6 hours before harvesting.

Preparation of Recombinant Proteins

A SmaI/HindIII restriction fragment encompassing amino acid residues from 11 to 192 of mGXII-1 PLA₂ was cloned into the EcoRV and HindlIl site of pET-29c (Novagene, Madison, Wisc.). The resulting plasmid and the empty pET-29c were used to transform the BL21 E. Coli (Novagene), respectively. One ml of overnight culture of the transformed BL-21 cells was diluted with 50 ml fresh LB medium containing kanamycin (30 μg/ml), cultured at 37° C. for three hours, and was induced with 1 mM IPTG for 2 hours to express recombinant proteins. The induced BL-21 cells were resuspended in 5 ml of 1× bind/wash buffer (20 mM Tris-HCl pH 7.5, 0.15 M NaCl, 0.1% Triton X-100) and sonicated three times (10 seconds each time) on ice. The insoluble fractions were solubilized in 6M guanidine hydrochloride on ice for one hour, dialyzed against at least 2 liters of PBS. The recombinant protein and the control bacterial extract thus prepared were used for phospholipase A2 enzymatic assays.

Characterization of Phospholipase A2 Activity

PLA₂ activity was assessed by the hydrolysis of 1-palmitoyl-2-[¹⁴C]arachidonyl-phosphotidylethanolamine to liberate [¹⁴C]arachidonic acid using a liposome-based assay. A 30 μl sample of recombinant mGXII-1 PLA₂ at approximately 80 ng/μl or control bacterial extract was adjusted to a final volume of 125 μl containing 4 mM CaCl₂, 10 mM Tris-HCl, pH 8.5, and 3.6 μM 1-palmitoyl-2-[¹⁴C]arachidonoyl-phosphotidylethanolamine for 1 hour at 37° C. The reaction was stopped by the addition of 625 μl of Dole's reagent. Free [¹⁴C]arachidonic acid was extracted in n-heptane and counted in a liquid β-scintillation counter. The pH dependence of the enzyme was determined using Tris-HCl as buffer over a pH range from 6.5 to 9.0 in 4 mM CaCl₂. To determine the Ca²⁺ dependence of the enzyme, enzymatic activity was initially assessed in the absence of Ca²⁺ from the bacterial extract. All enzymatic activity was lost in 300 μM EDTA. Recombinant enzyme was then assayed as described above at pH 8.5, with increasing concentration of CaCl₂, in the presence of 300 μM EDTA. The capacity of DTT to inhibit PLA₂ activity was assessed in the presence of 0.1% BSA. Substrate specificity was assessed using the following phospholipids: 1-palmitoyl-2-[¹⁴C]arachidonoyl-phosphatidylethanolamine, 1-palmitoyl-2-[¹⁴C]arachidonoyl-phophatidylcholine, 1-stearoyl-2-[L¹⁴C]arachidonoyl-phophotidylinositol, and 1-palmatoyl-2-[¹⁴C]palmitoyl-phosphotidylcholine, purchased from NEN; and 1-stearoyl-2-[¹⁴C]arachidonoyl-phosphatidylcholine, and 1-palmitoyl-2-[¹⁴C]linoleoyl-phophatidylethanolamine, purchased from Amersham. Porcine pancreatic PLA₂ was used as a positive control in experiments to determine substrate specificity and inhibition by DTT.

Northern Analysis and RT-PCR

Stimulated or unstimulated cells were harvested and total RNA was prepared by using the Trizol reagent (GIBCO RBL, Gaithersburg, Md.) according to the manufacturer's instructions. Total RNA derived from various organs was purchased from Ambion (Austin, Tex.). For Northern analysis, ten microgram of each RNA sample was fractionated on a 1.2% agarose gel, transferred to a nitrocellulose membrane and hybridized with indicated cDNA probes in the QuickHyb buffer (Stratagene, La Jolla, Calif.). The cDNA probes used were the full length mGXII-1 PLA₂, the full length mGV PLA₂, and the full length γ-actin. RT-PCR was performed by using the RT-PCR kit (Promega, Madison, Wis.) according to the manufacturer's suggestions. 0.5 ∥g of each RNA sample was used per RT-PCR reaction. The sequences of primers used in RT-PCR are mGXII-1 sense: 5′-GGGCAGGAACAGGACCAGACCACCG-3′ (SEQ ID NO:11); mGXII-2 sense: 5′-CCACAGTGGTCCTGGGAAGTACTGGG-3′ (SEQ ID NO:12); mGXII-1 and mGXII-2 antisense: 5′-GGTTTATATCCATAGCGTGGAACAGGCTTCG-3′ (SEQ ID NO:13); mGV sense: 5′-GTCCATGATTGAGAAGGTGACC-3′ (SEQ ID NO: 14); mGV antisense: 5′-TCATAGGACTGGGTCCGAATGG-3 (SEQ ID NO:15); mGX sense: 5′-GGTCACATGTATACAAGCGTGG-3′ (SEQ ID NO:16); mGX antisense: 5′-ATGTGATGGTCCATGCACTTCC-3′ (SEQ ID NO:17); β-actin sense: 5′-GTGGGCCGCTCTAGGCACCA-3′ (SEQ ID NO:18); β-actin antisense: 5′-CGGTTGGCCTTAGGGTTCAGGGGGG-3′ (SEQ ID NO:19).

GFP Fusion Protein Analysis

A partially digested SalI/BglII fragment and a partially digested XhoI/BglII cDNA fragment encoding nearly full-length cDNAs of mGXII-1 PLA₂ and mGXII-2 PLA₂, respectively, were cloned into the XhoI/BamHI site, in frame with the GFP cDNA, of pEGFP-N (Clontech, Palo Alto, Calif.). The resulting expression vectors and empty pEGFP-N vector were used to transfect BHK cells by using Effectene (Qiagen, Valencia, Calif.) according to the manufacturer's instructions. The transfected cells were replated on glass slides twenty-four hours later, rested overnight, fixed with 3% paraformaldehyde for 10 minutes at 4° C., stained with DAPI (0.005% in PB) for 2 minutes at room temperature, mounted with the by fluorescence microscopy.

Immunocytochemistry

The full length mGXII-1 PLA₂ cDNA was cloned into the SalI site of the pCI (Promega) vector. A SphI/NotI restriction fragment, encoding the last 12 amino acid residues of mGXII-1 PLA₂ was replaced with a double-stranded oligonucleotide encoding an HA tag. The resulting vector, and an expression vector for HA-tagged NIP45 as a control, were used to transfect BHK cells. The transfected BHK cells were replated and fixed as described above. The fixed BHK cells were then permeabilized with acetone (2 minutes at −20° C.), stained with anti-HA antibody (1:500 dilution of 12CA5, Boehringer Mannheim, Indianapolis, Ind.), washed three times with PBS, and restained with Cy3-conjugated goat anti-mouse IgG (H+L) antibody (1:300 dilution, Jackson ImmunoResearch Laboratories, West Grove, Pa.).

### EXAMPLE 1

### Molecular Cloning of Murine GXII PLA₂, a Novel Member of the PLA₂ Family

In the process of screening a D10 cDNA Th2 cell library, a hybrid cDNA clone was isolated that contained the 3′ untranslated region of the c-maf protooncogene fused to an approximately 1.5 kb novel cDNA. Sequence analysis revealed that the novel 1.5 kb cDNA encoded an open reading frame (ORF) of 191 amino acid residues, containing a signal peptide at its N-terminus and a conserved sequence for the catalytic domain of low molecular weight PLA₂ enzymes. The nucleotide sequence of the cDNA, and encoded amino acid sequence, are shown in SEQ ID NOs: 3 and 4, respectively.

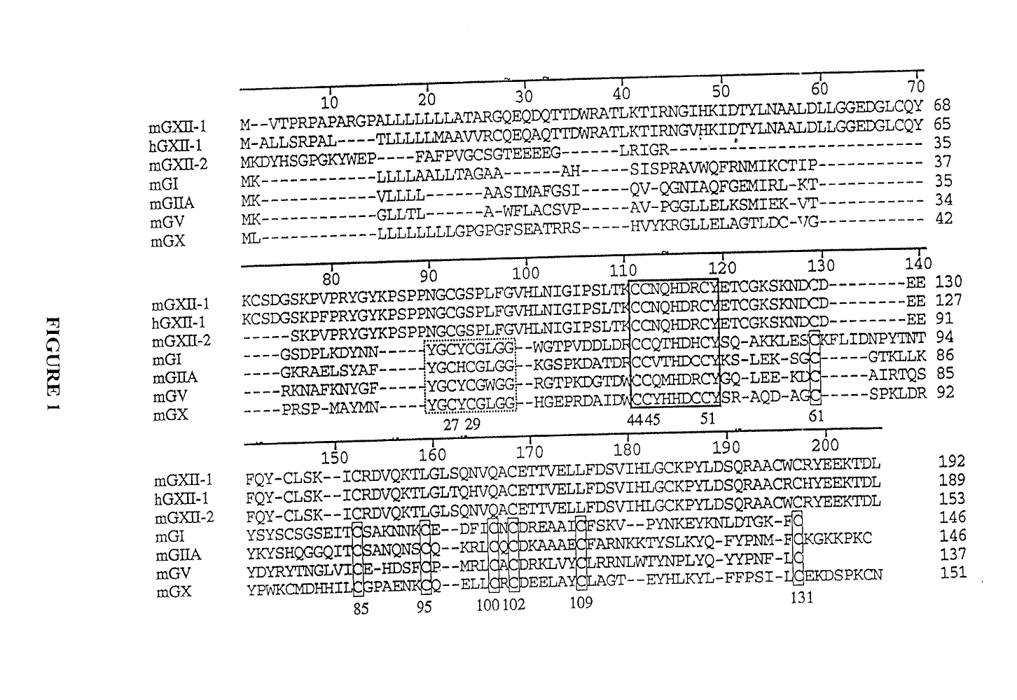

In addition, the ORF contained 14 cysteine residues similar to the GIIA PLA₂ protein. However, the position and spacing of the cysteine residues in this novel ORF were distinct from those of other low molecular weight PLA₂s. For instance, while this ORF contains cysteine residues that are capable of forming disulfide bonds 27-131 and 51-102, it lacks the appropriately spaced cysteine residues required to form disulfide bonds 29-45, 44-109, 61-95, and 85-100, which are found in all the mammalian low molecular weight PLA₂s. The position of spacing of cysteine residues in this novel ORF are also different from those of other histidine based PLA₂s, such as GIII, GIX, and GXI. This novel ORF also lacks a canonical calcium binding domain for low molecular weight PLA₂s. Furthermore, its predicted molecular weight is approximately 21 kDa which is significantly larger than that of the low molecular weight PLA₂s which are typically ˜14 kDa. These results implied that the ORF represented a novel member of the PLA₂ family, henceforth called group XII-1 PLA₂ (GXII-1 PLA₂). A comparison of the amino acid sequence of GXII-1 PLA₂ to other members of the PLA₂ family (e.g., mGI, mGIIA, mGV and mGX) is shown in FIG. 1.

### EXAMPLE 2

### Identification of Human Group XII-1 PLA₂ and an Alternatively Spliced Form of Murine Group XII-1 PLA₂

The Genbank database was searched with the mGXII-1 PLA₂ sequence and several human EST clones, including accession numbers AI189300, BE271295, BE271092 and BF111901 were identified that appeared to be related to mGXII-1 PLA₂. Using the human EST clone sequences and the mGXII-1 PLA₂ sequence as a template for comparison purposes, a composite human cDNA sequence was prepared that encodes the human GXII-1 PLA₂ (hGXII-1), which is more than 90% homologous to the mGXII-1 PLA₂ (see FIG. 1 for a comparison of the murine and human GX11-1 PLA₂ amino acid sequences). A further search of the Genbank database with the hGXII-1 PLA₂ sequence, revealed that the hGXII-1 PLA₂ sequence matched a genomic sequence derived from human chromosome 4q25 (accession number AC004067). Furthermore, a Drosophila ortholog (accession number AAF49567), which contains a consensus histidine catalytic PLA₂ domain and 14 cysteine residues with spacing almost identical to that of mGXII-1 PLA₂, was present in Genbank. This result suggests that GXII PLA₂ is evolutionarily conserved. In addition to hGXII-1 PLA₂, two murine EST clones (accession numbers AA008695 and AA204520) were identified encoding a mGXII-1 PLA₂-related open reading frame, in which the first 73 amino acid residues of mGXII-1 PLA₂, including the signal peptide, were replaced with a novel amino acid sequence of 34 residues following a distinct initial methionine (FIG. 1). This related open reading frame, independently derived from embryo and lymph node cDNA libraries, does not encode any obvious signal peptide and might represent an alternatively spliced form of mGXII-1 PLA₂; and was designated as murine group XII-2 PLA₂ (mGXII-2 PLA₂).

### EXAMPLE 3

### Characterization of Recombinant mGXII-l Phospholipase A₂ Activity

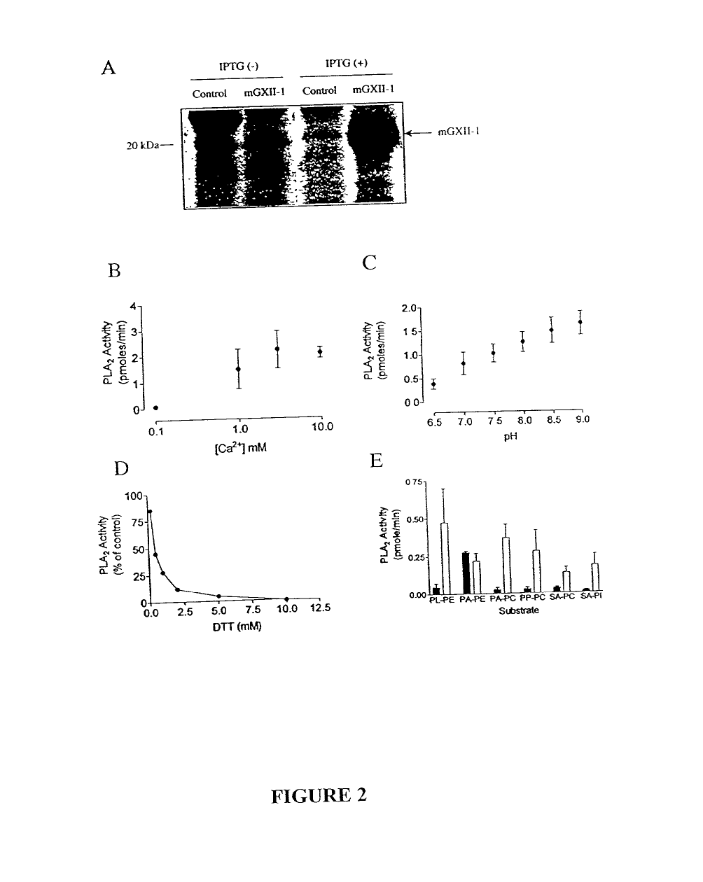

To determine if mGXII-1 PLA₂ has functional phospholipase A₂ catalytic activity, a near full length recombinant mGXII-1 PLA₂ was generated, which is approximately 20 kDa as analysed on a SDS-polyacrylamide gel (FIG. 2A). The capacity of the recombinant protein to hydrolyze arachidonic acid in the sn-2 position of phosphatidylethanolamine in a liposome presentation was determined. Maximal phospholipase A₂ activity was observed at basic pH in millimolar calcium; there was no detectable PLA₂ activity at submillimolar Ca²⁺ concentration (FIGS. 2B & 2C). PLA₂ activity was inhibited by DTT (FIG. 2D). An assessment of substrate specificity revealed a marked preference for arachidonic acid in the sn-2 position of phosphatidylethanolamine, in contrast to GIB PLA₂ from porcine pancreas that showed no such specificity (FIG. 2E).

### EXAMPLE 4

### Subcellular Localization of mGXII PLA₂ Enzymes

Similar to other low molecular weight PLA₂s, the mGXII-1 PLA₂ contains a signal peptide at its N-terminus, suggesting that mGXII-1 PLA₂ might be a secreted protein or a membrane associated protein. To determine their subcellular localization, both mGXII-1 PLA₂ and mGXII-2 PLA₂ were fused with green fluorescent protein (GFP) and then overexpressed in BHK cells. BHK cells were transfected with expression vectors for GFP, GFP/mGXII-1 PLA₂, or GFP/mGXII-2 PLA₂ fusion protein, fixed with paraformaldehyde, stained with DAPI, and observed under fluorescence microscopy. The results showed that mGXII-1 PLA₂ displayed a pattern consistent with localization to the Golgi/ER in BHK cells. In a second set of experiments, BHK cells were transfected with expression vectors for HA-tagged NIP45 or HA-tagged mGXII-1 PLA₂ and subjected to immunocytochemistry by using an anti-HA antibody as described above regarding Materials and Methods. The Golgi/ER pattern of mGXII-1 PLA₂ was more obvious when a HA-tagged mGXII-1 PLA₂ was used. As controls, overexpression of the GFP expression plasmid alone displayed a diffuse pattern and a HA-tagged nuclear protein, NIP45, was localized to the nuclei of BHK cells. In contrast to mGXII-1 PLA₂, overexpression of GFP/mGXII-2 PLA₂ gave a pattern indistinguishable with that of GFP alone. This is in agreement with the absence of a signal peptide in mGXII-2 PLA₂. These results demonstrated that mGXII-1 PLA₂ and mGXII-2 PLA₂ have distinct subcellular localizations.

### EXAMPLE 5

### Group XII PLA₂s and Group VPLA₂ are Preferentially Expressed in Th2 Cells

Since there is some evidence to suggest that the PLA₂/COX/eicosanoid cascade regulates the differentiation and function of helper T cells, the expression of mGXII PLA₂s was examined among various Th clones by Northern analysis using a cDNA probe common to both mGXII-1 PLA₂ and mGXII-2 PLA₂. Preparation of the two Northern blots shown in FIGS. 3A and 3B was as described previously (see Ho, I-C. et al. (1996) Cell 85:973-983; Miaw, S. C. (2000) Immunity 12:323-333). Both blots were stripped prior to hybridization with the mGXII PLA₂ cDNA probe. As shown in FIG. 3A, the expression of mGXII PLA₂s could be easily detected in Th2 clones, such as D10 and CDC35, and was further induced upon stimulation with anti-CD3. In contrast, the Th1 clones expressed very low levels of mGXII PLA₂s which were also less abundant after anti-CD3 stimulation than transcript levels in Th2 clones. Furthermore, mGXII PLA₂ was expressed at very low levels in a naïve CD4⁺Th cell population and was dramatically induced in cells that had been polarized along the Th2 pathway but not the Thl pathway (FIG. 3B). Of note, two transcripts were detected by the common cDNA probe, further suggesting the presence of alternative splice forms or homologues of mGXII PLA₂.

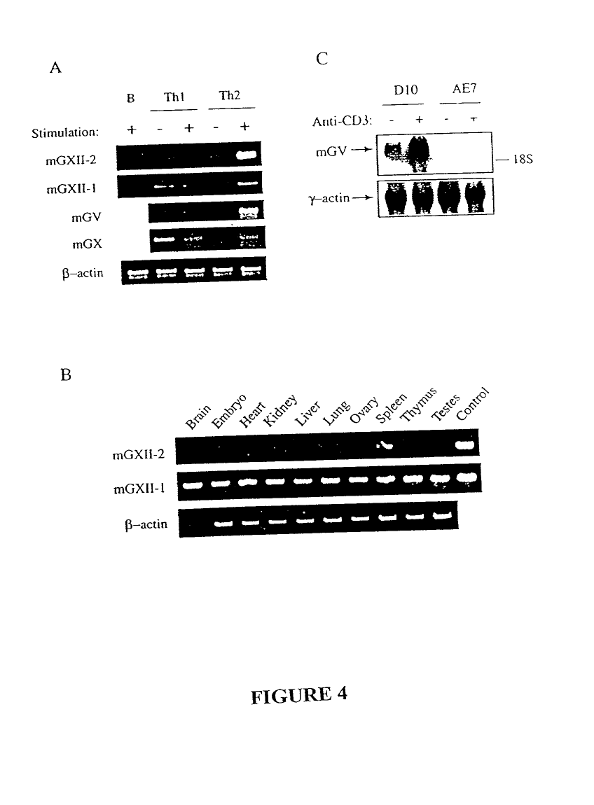

Since we were not able to distinguish mGXII-1 PLA₂ and mGXII-2 PLA₂ by Northern analysis, sequence-specific primers were used in RT-PCR to examine the expression and cell type-specificity of both forms of mGXII PLA₂. As shown in FIGS. 4A and 4B, the expression kinetics and cell type-specificity of both mGXII-1 PLA₂ and mGXII-2 PLA₂, in particular mGXII-2 PLA₂, as examined by RT-PCR, are similar to those revealed by Northern analysis. Importantly, mGXII-2 PLA₂ is almost exclusively expressed in stimulated normal Th2 but not Th1 or B cells (FIG. 4A).

In contrast to its subset-specific expression among the Th cell lineages, transcripts encoding mGXII-1 PLA₂ could be detected in a variety of organs and tissue (FIG. 4B). Interestingly, transcripts encoding mGXII-2 PLA₂ were only detected in spleen and, to a much lesser degree, in embryo (FIG. 4B). This is in agreement with the origin of the two mGMII-2 PLA₂ EST clones described previously.

The knowledge that an arachidonic acid selective PLA₂, GIV PLA₂, could function in concert with GV PLA₂ for PGE2 generation in a mouse macrophage line (Shinohara, H. et al (1999) J. Biol. Chem. 274:12263-12268) and for PGD2 production by mouse bone marrow derived mast cells (Reddy, S. T. et al. (1997) J. Biol. Chem. 272:13591-13596; Bingham, C. O. (1999) J. Biol. Chem. 274:31476-31484), prompted an assessment of the expression of GrV PLA₂ in Th cells. The RT-PCR analysis above was repeated using primers specific to murine GV (mGV) PLA₂ or murine GX (mGX) PLA₂. As shown in FIG. 4A, the expression of mGV PLA₂ could be detected in stimulated Th2 cells but not in Thl cells, whereas the expression of mGX PLA₂ was comparable between Th1 and Th2 cells. The Th2 cell-specific expression of mGV PLA₂ was further confirmed by Northern analysis using Th clones. As shown in FIG. 4C, mGV PLA₂ was expressed at a very low level in resting D10 cells (Th2 cells), however, its expression was dramatically induced within 6 hours after anti-CD3 stimulation. In contrast, no expression of mGV PLA₂ was detected in AE7 cells (Th1 cells).

### EXAMPLE 6

### Generation and Characterization of GXII PLA₂ Deficient Mice

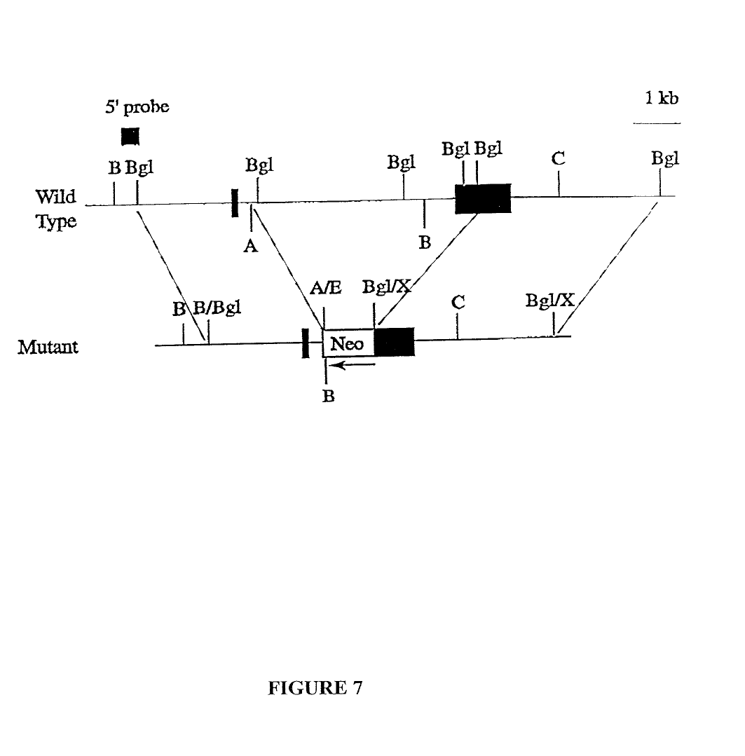

In order to study the function of GXII PLA₂, GXII PLA₂ deficient mice were generated by using standard gene ablation techniques, utilizing a gene “knockout” construct in which the neomycin gene was inserted into a portion of the mGXII PLA₂ gene. A schematic diagram of the wild type mGXII PLA₂ gene and the mutant allele created by homologous recombination is shown in FIG. 7. To identify homologous recombinant animals, Southern blot analysis of genomic DNA was performed. Ten micrograms of genomic DNA, prepared from mouse tails, was digested with BamHI restriction enzyme, fractionated on a 0.8% agarose gel, transferred to nitrocellulose membrane and hybridized with the 5′ probe illustrated in FIG. 7. The wild type genomic fragment was visualized as a 6 kb band, whereas the mutant BamHI restriction fragment was visualized as a 2.5 kb band. Heterozygous mice were identified by the presence of both a 6 kb and a 2.5 kb band, whereas the homozygous mutant mice were identified by the presence of only the 2.5 kb band.

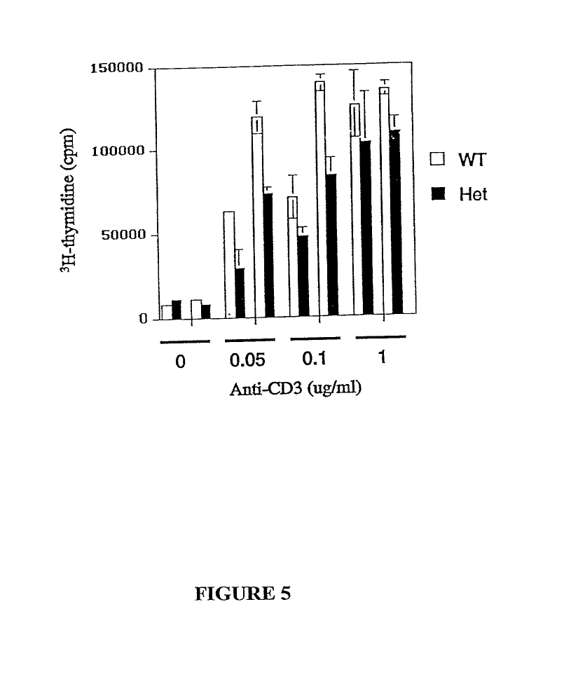

Preliminary data suggests that GXII PLA₂ is essential for the survival of embryo. Among approximately 50 live offspring from the interbreeding of GXII PLA₂(+/−) mice, none of them carries the genotype of GXII PLA₂(−/−). The embryonic lethal phenotype precludes the direct study of immune responses in the absence of GXII PLA₂. Thus, we chose to study T cells derived from GXII PLA₂(+/−) mice (i.e., heterozygous mice) in hopes of seeing a gene dose effect. The GXII PLA₂(+/−) mice are grossly normal. They have normal sized thymus, spleen, and lymph node; and the maturation of T and B cells derived from these mice is also normal except a slight decrease in the percentage of CD4 single positive thymocyte. The proliferation response of splenocytes was studied in the heterozygous mice and wildtype littermates. Splenocytes derived from GXII PLA₂(+/−) mice or wild type littermates were stimulated with ant-CD3 or LPS. Interestingly, in more than half of the experiments performed, splenocytes derived from GXII PLA₂(+/−) mice had a significant decrease (30-50%) of proliferation in response to anti-CD3 as compared to that of wild type splenocytes. In contrast, GXII PLA₂(+/−) splenocytes respond normally to LPS. Results from two independent experiments using anti-CD3 stimulation are summarized in FIG. 5, showing decreased proliferative responses in the heterozygous mice.

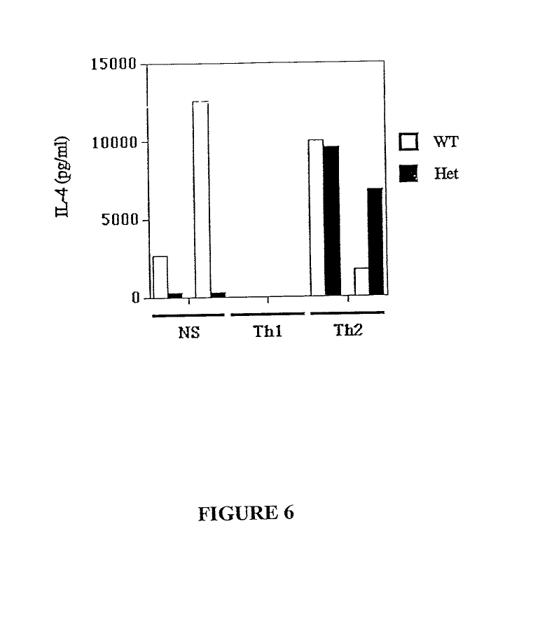

In peripheral Th cells, GXII PLA₂, in particular GXII-2 PLA₂, is preferentially expressed in Th2 cells. It is possible that GXII PLA₂ might be involved in the differentiation and effector function of Th cell. To address this question, peripheral CD4+Th cells were purified from GXII PLA₂(+/−) mice or wild type littermates, and stimulated in vitro with anti-CD3 under the non-skewing, Thl -skewing (IL-12 and anti-IL-4), or Th2-skewing (IL-4 and anti-IL-12) condition. The levels of cytokine produced by the differentiated Th cells were then examined by ELISA. The results are summarized in FIG. 6. Very interestingly, GXII PLA₂(+/−) Th cells, differentiated under non-skewing condition, produce significantly lower levels of IL-4 in comparison to those of wild type Th cells (see FIG. 6, first set of columns). The defect of IL-4 production can be compensated by exogenous IL-4, as GXII PLA₂(+/−) Th2 cells, differentiated under the Th2-skewing condition (in which exogenous IL-4 is added), produce normal levels of IL-4. Similar to the decreased proliferation phenotype described above, this defect of IL-4 production was seen in approximately 50% of GXII PLA₂(+/−) mice. This is not unexpected given that GXII PLA₂(+/−) Th cells can still produce variable levels of GXII PLA₂.

Taken together, this analysis on GXII PLA₂(+/−) mice suggests a critical role of GXII PLA₂ in regulating the differentiation and function of Th cells. This statement is further supported by the inducible expression of GXII PLA₂ in Th2 cells. Furthermore, the lethal phenotype seen in GXII PLA₂(−/−) embryos indicates that GXII PLA₂ have other functions outside the immune system and are unique among the families of phospholipase A2 enzyme.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. An isolated nucleic acid molecule comprising a nucleotide sequence encoding GXII PLA₂, or a biologically active portion thereof.

2. An isolated nucleic acid molecule comprising a nucleotide sequence encoding a protein, wherein: (i) the protein comprises an amino acid sequence at least 60% homologous to the amino acid sequence of SEQ ID NO: 2, SEQ ID NO: 4 or SEQ ID NO: 6, or (ii) a nucleotide sequence is at least 60% homologous to the nucleotide sequence of SEQ ID NO: 1, SEQ ID NO: 3 or SEQ ID NO: 5; and wherein the protein selectively hydrolyzes arachidonic acid in the sn-2 position of phosphatidylethanolamine.

3. The isolated nucleic acid molecule of claim 2, wherein the protein comprises an amino acid sequence at least 70% homologous to the amino acid sequence of SEQ ID NO: 2, SEQ ID NO: 4 or SEQ ID NO: 6 or the nucleotide sequence is at least 70% homologous to the nucleotide sequence of SEQ ID NO: 1, SEQ ID NO: 3 or SEQ ID NO:5.

4. The isolated nucleic acid molecule of claim 2, wherein the protein comprises an amino acid sequence at least 80% homologous to the amino acid sequence of SEQ ID NO: 2, SEQ ID NO: 4 or SEQ ID NO: 6 or the nucleotide sequence is at least 80% homologous to the nucleotide sequence of SEQ ID NO: 1, SEQ ID NO: 3 or SEQ ID NO: 5.

5. The isolated nucleic acid molecule of claim 2, wherein the protein comprises an amino acid sequence at least 90% homologous to the amino acid sequence of SEQ ID NO: 2, SEQ ID NO: 4 or SEQ ID NO: 6 or the nucleotide sequence is at least 90% homologous to the nucleotide sequence of SEQ ID NO: 1, SEQ ID NO: 3 or SEQ ID NO: 5.

6. An isolated nucleic acid molecule at least 30 nucleotides in length which hybridizes under stringent conditions to the nucleic acid molecule of SEQ ID NO: 1, SEQ ID NO: 3 or SEQ ID NO: 5.

7. The isolated nucleic acid molecule of claim 6 which comprises a naturally-occurring nucleotide sequence.

8. The isolated nucleic acid molecule of claim 6 which encodes a mouse GXII PLA₂.

9. The isolated nucleic acid molecule of claim 6 which encodes a human GXII PLA₂.

10. The isolated nucleic acid molecule of claim 6, which is at least 50 nucleotides in length.

11. The isolated nucleic acid molecule of claim 6, which is at least 100 nucleotides in length.

12. The isolated nucleic acid molecule of claim 6, which is at least 300 nucleotides in length.

13. The isolated nucleic acid molecule of claim 6, which is at least 500 nucleotides in length.

14. The isolated nucleic acid molecule of claim 6, which is at least 700 nucleotides in length.

15. An isolated nucleic acid molecule comprising the coding region of the nucleotide sequence of SEQ ID NO: 1, SEQ ID NO: 3 or SEQ ID NO: 5.

16. The isolated nucleic acid molecule of claim 15, comprising the nucleotide sequence of SEQ ID NO: 1, SEQ ID NO: 3 or SEQ ID NO: 5.

17. An isolated nucleic acid molecule encoding the amino acid sequence of SEQ ID NO: 2, SEQ ID NO: 4 or SEQ ID NO: 6.

18. An isolated nucleic acid molecule encoding a GXII PLA₂ fusion protein.

19. An isolated nucleic acid molecule which is antisense to a nucleic acid molecule encoding GXII PLA₂.

20. An isolated nucleic acid molecule which is antisense to the coding strand of the nucleic acid molecule of SEQ ID NO: 1, SEQ ID NO: 3 or SEQ ID NO: 5.

21. The isolated nucleic acid molecule of claim 20 which is antisense to a coding region of the coding strand of the nucleotide sequence of SEQ ID NO: 1, SEQ ID NO: 3 or SEQ ID NO: 5.

22. The isolated nucleic acid molecule of claim 20 which is antisense to a noncoding region of the coding strand of the nucleotide sequence of SEQ ID NO: 1, SEQ ID NO: 3 or SEQ ID NO: 5.

23. A vector comprising the nucleic acid molecule of claim 1.

24. The vector of claim 23, which is an expression vector.

25. The vector of claim 24, which encodes a protein comprising the amino acid sequence of SEQ ID NO: 2, SEQ ID NO: 4 or SEQ ID NO: 6.

26. The vector of claim 24, which comprises the coding region of the nucleotide sequence of SEQ ID NO: 1, SEQ ID NO: 3 or SEQ ID NO: 5.

27. A host cell containing the vector of claim 23.

28. A host cell containing the expression vector of claim 24.

29. A method for producing GXII PLA₂ protein comprising culturing the host cell of claim 28 in a suitable medium until GXII PLA₂ protein is produced.

30. The method of claim 29, further comprising isolating GXII PLA₂ protein from the medium or the host cell.

31. An isolated GXII PLA₂ protein or a biologically active portion thereof.

32. An isolated protein which comprises an amino acid sequence at least 60% homologous to the amino acid sequence of SEQ ID NO: 2, SEQ ID NO: 4 or SEQ ID NO: 6 and and wherein the protein selectively hydrolyzes arachidonic acid in the sn-2 position of phosphatidylethanolamine.

33. The isolated protein of claim 32, which is at least 70% homologous to the amino acid sequence of SEQ ID NO: 2, SEQ ID NO: 4 or SEQ ID NO: 6.

34. The isolated protein of claim 32, which is at least 80% homologous to the amino acid sequence of SEQ ID NO: 2, SEQ ID NO: 4 or SEQ ID NO: 6.

35. The isolated protein of claim 32, which is at least 90% homologous to the amino acid sequence of SEQ ID NO: 2, SEQ ID NO: 4 or SEQ ID NO: 6.

36. A fusion protein comprising a GXII PLA₂ polypeptide linked to a non-GXII PLA₂ polypeptide.

37. An antigenic peptide of GXII PLA₂ comprising at least 8 amino acid residues of the amino acid sequence shown in SEQ ID NO: 2, SEQ ID NO: 4 or SEQ ID NO: 6, the peptide comprising an epitope of GXII PLA₂ such that an antibody raised against the peptide forms a specific immune complex with GXII PLA₂.

38. An antibody that specifically binds GXII PLA₂.

39. A method for identifying a compound that modulates the activity of GXII PLA₂, comprising
providing an indicator composition comprising GXII PLA₂ and a substrate for GXII PLA₂;
contacting the indicator composition with a test compound; and
determining the effect of the test compound on GXII PLA₂ activity toward the substrate in the indicator composition to thereby identify a compound that modulates the activity of GXII PLA₂.

40. The method of claim 39, wherein the substrate is arachidonic acid in the sn-2 position of phosphatidylethanolamine.

41. A method for detecting the presence of GXII PLA₂ protein or MRNA in a biological sample, comprising contacting the biological sample with an agent capable of detecting GXII PLA₂ protein or mRNA such that the presence of GXII PLA₂ protein or mRNA is detected in the biological sample.

42. A method for modulating prostaglandin production by a Th2 cell comprising contacting the Th2 cell with a modulator of GXII PLA₂ or GV PLA₂ such that prostaglandin production by the cell is modulated.

43. A method for modulating Th2 cell differentiation or activity comprising contacting Th2 cells, or precursors thereof, with a modulator of GXII PLA₂ or GV PLA₂ such thatTh2 cell activity is modulated.
